(19) United States
(12) Patent Application Publication
(10) Pub. No.: US 2006/0165700 A1
(43) Pub. Date: Jul. 27, 2006

(54) Cancer therapy using whole glucan particles and antibodies

(76) Inventors: Gary R. Ostroff, Worcester, MA (US); Gordon D. Ross, Prospect, KY (US); Trunetta Jo Dockter Ross, Prospect, NY (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/526,175

(22) PCT Filed: Sep. 4, 2003
(86) PCT No.: PCT/US03/27841

§ 371 Date: Jul. 29, 2005

Related U.S. Application Data
(60) Provisional application No. 60/408,126, filed on Sep. 4, 2002.

(51) Int. Cl. A61K 39/395; A61K 31/716

(52) U.S. Cl. 424/155.1; 514/54

(57) ABSTRACT

The present invention relates to methods of using whole glucan particles and complement activating antibodies for antitumor therapy. Whole glucan particles enhance the tumoricidal activity of the innate immune system by binding to the C3 complement protein receptor CR3. This binding enhances innate immune system cytotoxicity, as well as stimulating the release of activating cytokines.

### RELATED APPLICATION

This application claims the benefit of U.S. Provisional Application No. 60/408,126, filed on Sep. 4, 2002. The entire teachings of the above application are incorporated herein by reference.

### GOVERNMENT SUPPORT

The invention was supported, in whole or in part, by grant Ro1CA86412 from National Institute for Health/National Cancer Institute and grant BC010287 from the Department of Defense, U.S. Army. The Government has certain rights in the invention.

### BACKGROUND OF THE INVENTION

Beta glucan is a complex carbohydrate, generally derived from several sources, including yeast, bacteria, fungi and plants (cereal grains). These sources provide β-glucans in a variety of mixtures, purities and structures. The structural diversity of β-glucan results from the different ways the glucose molecules are able to link yielding compounds with different physical properties and biological properties. For example, β(1,3) glucan derived from bacterial and algae is linear, making it useful as a food thickener. Lentinan (from Lentinus edodes, Basidiomycete family) is a high MW β-glucan with β(1,6) branches off of the (1,3) backbone every three residues. Schizophyllan (from Schizophyllum commune, Basidiomycetes family) is similar, but with shorter β (1,6) side chains. Beta-glucan from barley, oat, or wheat has mixed (1,3)- and (1,4)-β-linkages in the backbone, but no (1,6)-β branches, and is generally of high molecular weight. The frequency of side chains, known as the degree of substitution or branching frequency, regulates secondary structure and solubility. Beta glucan derived from yeast has a backbone chain of β(1-3) linked glucose units with a low degree of inter and intra-molecular branching through β(1-6) linkages. Based on extensive published research it is widely accepted that baker's yeast (Saccharomyces cerevisiae) is a preferred source of β (1,3)-glucan, based on the purity and activity of the product obtained.

The cell wall of S. cerevisiae is mainly composed of β-glucans, which are responsible for its shape and mechanical strength. While best known for its use as a food grade organism, yeast is also used as a source of zymosan, a crude insoluble extract used to stimulate a non-specific immune response. Yeast zymosan serves as a rich source of β (1,3) glucan. Yeast-derived beta 1,3 glucan appears to stimulate the immune system, in part, by activating the innate immune system as part of the body's basic defence against fungal infection. Yeast β (1,3) glucan is a polysaccharide composed primarily of β (1-3)-linked glucose molecules with periodic β (1-3) branches linked via β (1-6) linkages and is more formally known as poly-(1-6)-β-glucopyranosyl-(1-3)-β-D-glucopyranose. Glucans are structurally and functionally different depending on the source and isolation methods.

Beta glucan possesses a diverse range of activities. The ability of (β-glucan to increase nonspecific immunity and resistance to infection is similar to that of endotoxin. Beta glucan's activity as an immune adjuvant and hemopoietic stimulator compares to that of more complex biological response modifiers such as bacillus Calmette-Guerin and Corynebacteriun parvum. The functional activities of yeast β-glucans are also comparable to those structurally similar carbohydrate polymers isolated from fungi and plants. These higher molecular weight (1-3)-β-D-glucans such as schizophyllan, lentinan, krestin, grifolan, and pachyman exhibit similar immunomodulatory activities. Various preparations of both particulate and soluble β-glucans have been tested in animal models to elucidate the biological activities. The use of soluble and insoluble β-glucans alone or as vaccine adjuvants for viral and bacterial antigens has been shown to markedly increase resistance to a variety of bacterial, fungal, protozoan and viral infections. Beta glucan's hemopoietic effects include increased peripheral blood leukocyte counts and bone marrow and splenic cellularity, reflecting increased numbers of granulocyte-mactophage progenitor cells, splenic pluripotent stem cells, and erythroid progenitor cells, as well as increased serum levels of granulocyte-monocyte colony-stimulating factor (GM-CSF).

The molecular mechanism of action of β-glucan appears to involve specific β-glucan receptor binding sites on the cell membranes of immune cells such as neutrophils and macrophages. Mannans, galactans, α(1-4)-linked glucose polymers and β(1-4)-linked glucose polymers have no avidity for this receptor. Recent data suggests that CR3, the receptor for C3 complement protein, serves as a major receptor for β-glucans. Ligand binding to the β-glucan receptor results in complement activation, phagocytosis, lysosomal enzyme release, and prostaglandin, thromboxane and leukotriene generation. Most β-glucan preparations described in the prior art stimulate production of cytokines such as IL-1 and TNF, which are known to have antitumor activity.

The potential antitumor activity of β-glucans has been under investigation for about 30 years, as disclosed primarily in the Japanese pharmaceutical literature. Lentinan, for example, has been extensively investigated both in animal models at 1 mg/kg for 10 days and in clinical trials since the late 1970s for advanced or recurrent malignant lymphoma and colorectal, mammary, lung and gastric cancers. A recent review describes much of this work, which has focused on β-glucans isolated from mushrooms (Borchers, AT, et al., Mushrooms and Immunity, 221(4), 281 (1999)). This work indicates that the antitumor activity of polysaccharides isolated from mushrooms is largely mediated by T cells and macrophages that are activated by β-glucan. Oral β-glucan isolated from crude yeast and cereal grain preparations has demonstrated antitumor activity as well. These studies used crude β (1,3) glucan preparations that are mixtures of β (1,3) glucan along with other polysaccharides such as β-glucans, mannans, chitin/chitosan, β (1,4) glucans, nucleic acids, proteins, and lipids. The β (1,3) glucan content of these preparations is typically less than 50% by weight. The effectiveness of various glucans differs in their ability to elicit various cellular responses, particularly cytokine expression and production, and in their activity against specific tumors. It has been proposed that the antitumor mechanism of action of β-glucans involves macrophage simulation and subsequent release of inflammatory mediators such as IL-1, TNF, and prostaglandin E2 (Sveinbjørnsson et al., Biochem. Biophys. Res. Commun. 223(3), 643 (1996)).

The subtle changes associated with cancer development can lead to different expression of surface proteins, which can stimulate a weak response by the adaptive immune system. These changes in surface antigen expression also provide a target for treatment using selective monoclonal antibodies (mAbs) or antitumor vaccines. Monoclonal antibodies have been developed to target various proteins expressed in colon cancer, lymphoma, breast cancer, and acute leukemia, for example. The immune basis of the clinical tumor response to mAb includes direct cytotoxicity and induced immunity, in which antibody-dependent cell-mediated cytotoxicity and complement-mediated cytotoxicity are responsible for the direct killing of tumor cells. However, it has been noted that the increased complement activation mediated by natural or monoclonal antibodies often shows little effect on tumor growth due to the inherent resistance of tumors to complement-mediated cytotoxicity, a fact that has frequently made mAbs or vaccines to tumor antigens ineffective therapeutically.

An increasing awareness exists that the destruction of tumors by the immune system requires a combination of effector mechanisms, and that a single vaccine, cytokine, or biological response modifier is unlikely to be successful in a majority of patients. For example, vaccines may elicit immune cytotoxic T lymphocytes and/or humoral antibody responses, but both of these responses have shortcomings. Antibodies are frequently ineffective because normal host cell proteins such as DAF, MCP, and CD59 inhibit complement-mediated cytotoxicity, and iC3b-opsonization of tumors does not, on its own, recruit phagocytes or NK cells. Antibody-dependent cell-mediated immunity is thought to fail because the IgG density achieved on tumors is too low and antibody Fc fragment-mediated cytotoxicity is suppressed by NK cell recognition of tumor cell MHC class I. Cell-mediated immunity utilizing cytotoxic T lymphocytes has disadvantages as well, since tumors, as part of the metastatic process, often lose the major histocompatability complex molecules required for antigen presentation. A need exists for a combined antitumor immunological approach that will overcome these shortcomings.

### SUMMARY OF THE INVENTION

The present application discloses a method of antitumor therapy in which insoluble β-glucan is used with complement activating anti-bodies directed to tumor antigens to provide an antitumor effect. Insoluble beta (1,3) glucan (referred to herein also as whole glucan particles, WGP) enhances the tumoricidal activity of the immune system by binding to the C3 complement protein receptor, CR3. In a preferred embodiment, the method provides a synergistic effect. This synergism is derived, in part, from the ability of antibodies to selectively target tumor cells while β-glucan amplifies the normally weak or ineffective humoral response by using the C3 deposition induced by the antibodies to target tumor cells for recognition by innate immune cells bearing β-glucan primed CR3.

The present application discloses that the anti-cancer immune activities stimulated by tumor antigen-directed monoclonal antibody therapy and anti-tumor vaccines are augmented by whole glucan particles. Cell surface monoclonal antibodies and tumor vaccines stimulate a complex anti-tumor immune response involving non-specific and specific immune responses. The non-specific immune responses involve innate immune cell factors (e.g., the complement system) and cells (dendritic, monocyte, macrophage, neutrophil and NK cells). Tumor antigen-directed monoclonal antibodies and antibodies stimulated by tumor vaccines bind to the surface of tumor cells and target these cells for direct complement action and complement-mediated cytotoxicity.

A key part of the mechanism of antibody-mediated tumor cell killing involves recognition of the antibody-tumor antigen complex by C3 complement protein, forming the C3-antibody-tumor antigen complex. This complex is recognized by innate immune cells via CR3. Innate immune cells bearing CR3 receptor bind to tumor cells through the specific interaction between CR3 and C3-antibody-tumor cell antigen complex but do not see the tumor as foreign and consequently do not induce tumorcidal activities. When beta glucan binds to CR3, innate immune cells are stimulated to exert their tumoricidal activities. In the present invention, these innate immune cells are also stimulated by WGP in cancer therapies. Monocytes, macrophages, neutrophils and NK cells become activated upon β (1,3)-glucan binding to CR3 on their cell surface. The activation of these cells by β (1,3)-glucan-CR3 interactions enhances the C3-antibody-tumor cell antigen complex-targeted tumoricidal activities of these cells, resulting in synergistically enhanced tumor cell killing.

A novel aspect of this invention is the synergistic anti-tumor activity of WGP with cell surface monoclonal antibodies and/or tumor vaccine therapies. Beta (1,3) glucan in the form of WPG has the advantage of being readily prepared in high purity from any source of β (1,3)-glucan. Additionally, it has been found that WGP is orally bioavailable to the target site. The use of WPG as an antitumor agent has a number of important aspects. First, the use of highly pure WPG leads to higher activity with fewer side effects. Second, WGP's once degraded can bind to the lectin binding domain of CR3, thereby activating the tumoricidal activities of innate immune cells. By utilizing the targeted activation creating by complement depostion, β-glucan leads to enhanced tumor clearance by the immune system, both through direct cytotoxic effects and by localized cytokine-mediated recruitment of immune cells

The present application also discloses a method of suppressing or eliminating tumor cells by administering to a subject in need of treatment a therapeutically effective amount of whole glucan particles and antibodies targeted to antigens of the tumor cells. The antibodies are complement activating antibodies. In certain embodiments, the antibodies are IgG subclass I or IgG subclass III. The antibodies are can also be induced in a patient by administering an appropriate vaccine or can be provided directly by administering monoclonal or polyclonal antibodies, such as by intraveneous administration of a monoclonal antibody. Alternatively, the antibodies can be naturally present in an individual. In certain embodiments, the whole glucan particles and antibodies provide a synergistic antitumor effect. The insoluble β(1,3) glucans (whole glucan particles) are administered orally, parenterally, or by other methods known in the art.

In certain embodiments, a method of suppressing or eliminating tumor cells comprises administering to a subject in need of treatment a therapeutically effective amount of whole glucan particles and at least one complement activating anti-tumor antibody; wherein the combination of glucan and antibody suppresses or eliminates tumor cells. The antibody can be introduced via a vaccine or by direct administration of a monoclonal or polyclonal antibody. In particular embodiments, the antibody is selected from the group consisting of: trastuzumab, rituximab, cetuximab and combinations thereof. The whole glucan particles can be administered orally or parenterally. In certain embodiments the whole glucan particles can be from yeast, fungal source (e.g., mushroom), or plant source such as cereal grains, for example, barley.

In other embodiments, the whole glucan particles and antibody provide a synergistic antitumor effect.

Also described is the use of whole glucan particles and complement activating anti-tumor antibody for the manufacture of a medicament for use in treating a neoplastic cell, wherein the combination of glucan and antibody retards the growth of the cell.

In another embodiment, a method of treating a neoplastic cell comprises administering to said cell a therapeutically effective dose of whole glucan particles and a complement activating antibody specific to the neoplastic cell is described. In certain embodiments, the combination of glucan and antibody protects the host of a neoplastic cell and acts by retarding the rate of growth of the neoplastic cell and/or inhibiting the growth of the neoplastic cell and/or extending the survival time of a host of the neoplastic cell.

In other embodiments, a method of suppressing or eliminating tumor cells, comprises administering to a subject in need of treatment a therapeutically effective amount of whole glucan particles and at least one complement activating anti-tumor antibody; wherein the combination of glucan and antibody suppresses or eliminates tumor cells and the complement activating antibody is coated on tumor cells and activates complement via iC3b deposition on the tumor cells. The whole glucan particle is taken up by macrophages, degraded and the degraded fragments bind to neutrophils in the bone marrow and through chemotaxis migrate and bind to antibody coated tumor cells where complement has been activated via iC3b deposited the tumor cells.

Also described is a method of suppressing or eliminating tumor cells, comprising administering to a subject in need of treatment a therapeutically effective amount of insoluble whole glucan particles wherein the whole glucan particles is taken up by macrophages, degraded and the degraded fragments bind to neutrophils in the bone marrow and through chemotaxis migrate and bind to antibody coated tumor cells where complement has been activated via iC3b deposited the tumor cells by a naturally occurring complement activating antibody, wherein the binding of glucan to the iC3b tumor cells results in suppressing or eliminating the tumor cells.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a drawing showing that activation of CR3 by C3-opsonized yeast requires both iC3b ligation and β-glucan attachment to the lectin site.

FIG. 2 is a drawing showing that bacteria lacking β-glucans do not trigger phagocytosis or degranulation via CR3.

FIG. 3 is a drawing showing that whole glucan particles degraded to soluble glucan binds to CR3 and primes the receptor to trigger degranulation and destructuion of bacteria or tumor cells targeted with iC3b.

FIG. 4 is a graph showing that β-glucan priming of murine neutrophil CR3 allows subsequent cytotoxic triggering by iC3b-opsonized breast tumor cells.

FIGS. 5A-5D are a series of graphs showing the two-color flow cytometric analysis of tumor cells from a patient with mammary carcinoma for IgM, IgG, or C3. FIG. 5A is a graph of anti mouse-IgG-PE versus Mlg-FITC. FIG. 5B is a graph of anti-MUC1-PE verses anti-IgM-FITC. FIG. 5C is a graph of anti-MUC1-PE verses anti-IgG-FITC. FIG. 5D is a graph of anti-MUC1 verses anti-C3-FITC.

FIG. 6 is a graph showing that suspensions of freshly excised primary mammary tumor cells bear sufficient C3 for cytotoxic recognition by allogeneic NK cells bearing β-glucan primed CR3.

FIG. 7 is a graph showing the results of β-glucan therapy of Ptas64 mammary carcinoma in Balb/c Mice.

FIG. 8 is a graph showing the failure of β-glucan tumor therapy in mice deficient in serum C3 or leukocyte CR3.

FIG. 9 is a graph showing the enhancement of anti-tumor mAb therapy of hepatic EL-4 lymphoma with β-glucan.

FIG. 10 is a graph showing the synergy of oral soluble barley β-glucan therapy with antibody against human LAN-1 neuroblastoma in nude mice.

FIG. 11 is a graph showing the results of therapy of Balb/c mammary carcinoma with i.v. mAb plus oral yeast insoluble β-glucan particles (WGP).

FIG. 12 is a graph showing that leukocyte recruitment via leukotriene B4 receptors is required for the tumoricidal activity of mAb plus oral WGP therapy.

FIG. 13 is a graph showing β-glucan and CR3-dependent stimulation of NK cell secretion of TNF-α

FIGS. 14A and 14B are graphs showing orally administered yeast whole β-glucan particles (WGP) enhance tumor regression (FIG. 14A) as measured by tumor diameter versus days after tumor implantation and survival (FIG. 14B) in a similar manner as i.v. yeast β-glucan.

FIGS. 15A-15D are a series of graphs showing tumor regression with orally administered soluble barley or particulate yeast β-glucan requires leukocyte CR3. FIG. 15A is a graph of tumor diameter (mm) versus days of therapy in wild-type mice. FIG. 15B is a graph of percent survival versus days of therapy in wild-type mice. FIG. 15C is a graph of tumor diameter (mm) versus days of therapy in CR3-deficient mice. FIG. 15D is a graph of survival verses days of therapy in CR3 deficient mice.

FIGS. 16 A-16D are a series of graphs showing long-term tumor-free survival with soluble barley or particulate yeast β-glucan therapy requires leukocyte CR3. FIG. 16A is a graph of tumor diameter (mm) versus days of therapy in wild-type mice. FIG. 16B is a graph of percent survival versus days of therapy in wild-type mice. FIG. 16C is a graph of tumor diameter (mm) versus days of therapy in CR3-deficient mice. FIG. 16D is a graph of survival verses days of therapy in CR3 deficient mice.

FIGS. 17A-17D are a series of graphs showing tumor regression with orally administered soluble barley or particulate yeast β-glucan requires serum C3. FIG. 17A is a graph of tumor diameter (mm) versus days of therapy in wild-type mice. FIG. 17B is a graph of percent survival versus days of therapy in wild-type mice. FIG. 17C is a graph of tumor diameter (mm) versus days of therapy in CR3-deficient mice. FIG. 177D is a graph of survival verses days of therapy in CR3 deficient mice.

FIGS. 18A-18D are a series of graphs showing tumor-free survival with orally administered soluble barley or particulate yeast β-glucan requires serum C3. FIGS. 18A-18B are graphs showing percent survival versus days after tumor implantation for wild type mice. FIGS. 18C-18D are graphs showing percent survival versus days after tumor implantation for CR3 deficient mice.

FIG. 19 is a graph showing uptake of soluble β-glucan by the marginated granulocyte pool requires membrane surface CR3.

### DETAILED DESCRIPTION OF THE PREFERRED EMBODIMENTS

The present application discloses a method of antitumor therapy in which β-glucan is used along with antibodies directed to tumor antigens to provide an antitumor effect on neoplastic cells. The preparation and methods of use of these glucans in combination with antibody for the treatment of neoplastic cells is described below.

As described herein, orally administered insoluble β-glucans were shown to elicit a tumoricidal activity. The glucan was able to prime CR3 (iC3b-receptor; CD11b/CD18) of circulating neutrophils to kill tumors opsonized with iC3b via monoclonal antibodies (mAbs). As described herein, oral barley β(1→3)(1→4) and whole glucan particles significantly enhanced tumor regression and survival mediated by anti-tumor mAbs. Tumor regression mediated by oral β-glucans did not occur in CR3-(CD11b^{-/-}) or C3-deficient mice, highlighting the requirement for leukocyte CR3 to recognize tumor-bound iC3b. The in vivo fate of oral whole glucan particles (WGP-F) was explored using WGP-fluorescein (WGP-F). Gastrointestinal macrophages shuttled orally absorbed WGP-F to lymphoid tissues, with WGP-F appearing within splenic macrophages 3 days after oral administration and within bone marrow (BM) macrophages after 5 days. CR3 was not required for macrophage uptake of WGP-F, as BM macrophages from both wild-type and CD11b^{-/-} mice contained WGP-F. BM macrophages digested WGP-F and secreted soluble β-glucan-F that was taken up by BM neutrophils via CR3. Only neutrophils elicited from mice that had been given oral WGP were able to kill iC3b-opsonized tumor cells. Macrophages shuttle oral β-glucans to the bone marrow where they prime neutrophil CR3 for tumoricidal activity.

Whole glucan particle (WGP) is a purified, yeast cell wall preparation. Whole glucan particles are produced by removing the mannan protein outer layer and exposing the β-glucan while retaining glucan's in vivo morphology. In certain embodiments, the whole glucan particles have a particle size of 1 micron or greater.

Another form of βglucan is an insoluble particle known as whole glucan particles (WGP). Whole glucan particles are the remnants of the yeast cell wall prepared by separating growing yeast from its growth medium and subjecting the intact cell walls of the yeast to alkali, thus removing unwanted proteins and nucleic acid material. In certain embodiments, what remains is a spherical beta-glucan particle with the outer mannan protein removed. Whole glucan particles may be obtained from any glucan-containing fungal cell wall source, but the preferred source is a strain of S. cerevisiae. In certain embodiments, the glucan content of preparations are greater than 50% glucan. In certain embodiments, the remainder can be comprised of intracellular lipids and/or glycogen. These insoluble particles have been shown to enhance host resistance to a wide range of infections, increase antibody production (adjuvant activity), increase leukocyte mobilization, and enhance wound healing. Methods of producing WGP are known in the art and are disclosed in U.S. Pat. Nos. 4,810,646, 4,492,540, 5,037,972, 5,082,936, 5,250,436, and 5,506,124, the contents of which are incorporated herein by reference in their entirety.

Microparticulate glucan particles are defined herein to be portions of whole glucan particles that result from finely grinding yeast cell wall β(1-3; 1-6) glucan down to a particle size of about 1 micron or less.

Various forms of particulate β-glucans have been prepared. One example is microparticulate whole glucan particles, which can be formed by finely grinding yeast cell wall β(1-3;1-6) glucan down to a particle size of about 1 micron or less. Beta glucan in this form has been described for use as a nutritional supplement and skin restorer, such as disclosed in U.S. Pat. No. 5,702,719, by Donzis. Other particulate glucans useful in the methods described herein, are WGP™ Beta Glucan and BetaRight™ obtained from Biopolymer Engineering, Inc., Eagan, Minn. Microparticulate β-glucan have also been shown to enhance the host's immune system. See U.S. Pat. Nos. 5,223,491 and 5,576,015, the teachings of which are incorporated herein by reference in their entirety.

In certain embodiments, whole glucan particles for use in the methods described herein are oral bioavailable formulations. “Bioavailable”, as used herein, means the whole glucan particle is able to reach the target of action. In other words, whole glucan particles have enough β (1,3; 1,6) glucan exposed for Peyer's patch uptake of the glucan. The glucan is taken up in the Peyer's patch and engulfed and degraded by macrophages, transported to the bone marrow where the degraded fragments are released. The degraded fragments bind to neutrophils in the bone marrow and through chemotaxis migrate to and bind to antibody coated tumors where complement has been activated via iC3b deposited on tumors. For example, the WGP is able to reach and act on tumor cells in combination with the antibody. At the site of action, the glucan acts to stimulate cells as a result of the binding or association of the glucan to the CR3 receptor that in turn primes or promotes the CR3 for action. The bioavailability of oral WGP is mediated by the transport of WGP to the bone marrow by gastrointestinal macrophages that degrade the particle. The degraded particles then function at the bone marrow as stimulators of neutrophils via CR3 activation when the neutrophils migrate to tumor cells and bind to iC3b on tumors.

Preparation of WGP glucan

Briefly, the process for producing whole glucan particles involves the extraction and purification of the alkali-insoluble whole glucan particles from the yeast or fungal cell walls. This process yields a product, which maintains the morphological and structural properties of the glucan as, found in vivo, as is referred to as a whole glucan, or whole glucan particles.

The structure-function properties of the whole glucan preparation depend directly on the source from which it is obtained and also from the purity of the final product. The source of whole glucan can be yeast or other fungi, or any other source containing glucan having the properties described herein. In certain embodiments, yeast cells are a preferred source of glucans. The yeast strains employed in the present process can be any strain of yeast, including, for example, S. cerevisiae, S. delbrueckii, S. rosei, S. microellipsodes, S. carlsbergensis, S. bisporus, S. fermentati, S. rouxii, Schizosaccharomyces pombe, Kluyveromyces polysporus, Candida albicans, C. cloacae, C. tropicalis, C. utilis, Hansenula wingei, H. arni, H. henricii, H. americana, H. canadiensis, H. capsulata, H. polymorpha, Pichia kluyveri, P. pastoris, P. polymorpha, P. rhodanensis, P. ohmeri, Torulopsis bovin, and T. glabrata.

Generally, the above procedure can be used to prepare and isolate other mutant yeast strains with other parent stains as starting material. Additionally, mutagens can be employed to induce the mutations, for example, chemical mutagens, irradiation, or other DNA and recombinant manipulations. Other selection or screening techniques may be similarly employed.

The yeast cells may be produced by methods known in the art. Typical growth media comprise, for example, glucose, peptone and yeast extract. The yeast cells may be harvested and separated from the growth medium by methods typically applied to separate the biomass from the liquid medium. Such methods typically employ a solid-liquid separation process such as filtration or centrifugation. In the present process, the cells are preferably harvested in the mid-to late logarithmic phase of growth, to minimize the amount of glycogen and chitin in the yeast cells. Glycogen, chitin and protein are undesirable contaminants that affect the biological and hydrodynamic properties of the whole glucan particles.

Preparation of whole glucan particles involves treating the yeast with an aqueous alkaline solution at a suitable concentration to solubilize a portion of the yeast and form an alkali-hydroxide insoluble whole glucan particles having primarily β(1-6) and β(1-3) linkages. The alkali generally employed is an alkali-metal hydroxide, such as sodium or potassium hydroxide or an equivalent. The starting material can comprise yeast separated from the growth medium. It is more difficult to control consumption of the aqueous hydroxide reactants and the concentration of reactants in the preferred ranges when starting with yeast compositions that are less concentrated. The yeast should have intact, unruptured cell walls since the preferred properties of the instant whole glucan particles depend upon an intact cell wall.

The yeast are treated in the aqueous hydroxide solution. The intracellular components and mannoprotein portion of the yeast cells are solubilized in the aqueous hydroxide solution, leaving insoluble cell wall material which is substantially devoid of protein and having a substantially unaltered three dimensional matrix of β(1-6) and β(1-3) linked glucan. The preferred conditions of performing this step result in the mannan component of the cell wall being dissolved in the aqueous hydroxide solution. The intracellular constituents are hydrolyzed and released into the soluble phase. The conditions of digestion are such that at least in a major portion of the cells, the three dimensional matrix structure of the cell walls is not destroyed. In particular circumstances, substantially all the cell wall glucan remains unaltered and intact.

In certain embodiments, the aqueous hydroxide digestion step is carried out in a hydroxide solution having initial normality of from about 0.1 to about 10.0. Typical hydroxide solutions include hydroxides of the alkali metal group and alkaline earth metals of the Periodic Table. The preferred aqueous hydroxide solutions are of sodium and potassium, due to their availability. The digestion can be carried out at a temperature of from about 20° C. to about 121° C. with lower temperatures requiring longer digestion times. When sodium hydroxide is used as the aqueous hydroxide, the temperature can be from about 80° C. to about 100° C. and the solution has an initial normality of from about 0.75 to about 1.5. The hydroxide added is in excess of the amount required, thus, no subsequent additions are necessary.

Generally from about 10 to about 500 grams of dry yeast per liter of hydroxide solution is used. In certain embodiments, the aqueous hydroxide digestion step is carried out by a series of contacting steps so that the amount of residual contaminants such as proteins are less than if only one contacting step is utilized. In certain embodiments, it is desirable to remove substantially all of the protein material from the cell. Such removal is carried out to such an extent that less than one percent of the protein remains with the insoluble cell wall glucan particles. Additional extraction steps are preferably carried out in a mild acid solution having a pH of from about 2.0 to about 6.0. Typical mild acid solutions include hydrochloric acid, sodium chloride adjusted to the required pH with hydrochloric acid and acetate buffers. Other typical mild acid solutions are in sulfuric acid and acetic acid in a suitable buffer. This extraction step is preferably carried out at a temperature of from about 20° C. to about 100° C. The digested glucan particles can be, if necessary or desired, subjected to further washings and extraction to reduce the protein and contaminant levels. After processing the product pH can be adjusted to a range of about 6.0 to about 7.8.

By conducting this process without a step of disrupting the cell walls, the extraction can be conducted at more severe conditions of pH and temperature than was possible with the prior art procedure that included a step of disrupting the cell walls. That is, the process of this invention avoids product degradation while employing these severe extraction conditions which permits elimination of time-consuming multiple extraction steps.

After the above aqueous hydroxide treatment step, the final whole glucan product comprises about 5 to about 30 percent of the initial weight of the yeast cell, preferably the product is from about 7 to about 15 percent by weight.

The aqueous hydroxide insoluble whole glucan particles produced is as set forth in the summary of the invention. The whole glucan particles can be further processed and/or further purified, as desired. For example, the glucan can be dried to a fine powder (e.g., by drying in an oven); or can be treated with organic solvents (e.g., alcohols, ether, acetone, methyl ethyl ketone, chloroform) to remove any traces or organic-soluble material, or retreated with hydroxide solution, to remove additional proteins or other impurities that may be present.

In certain embodiments, the whole glucan particles obtained from the present process are comprised of pure glucan, which consists essentially of β(1-6) and β(1-3) linked glucan. The whole glucan particles contain very little contamination from protein and glycogen. In certain embodiments, the whole glucan particles are spherical in shape with a diameter of about 2 to about 10 microns and contain greater than about 85% by weight hexose sugars, (or in other embodiments greater than about 60% hexose sugars), approximately 1% by weight protein and less than 1% of a detectable amount of mannan, as determined monosaccharide analysis or other appropriate analysis. Glucans obtained by prior processes contain substantially higher quantities of chitin and glycogen than the present glucans.

The second step as set forth above, involves the modification of the whole glucan particles, as produced above, by chemical treatment to change the properties of the glucan. It is contemplated that whole glucan particles derived from any yeast strain may be used, in addition to those particular strains described herein. As mentioned above, a very broad spectrum of yeast or mutant yeast strains may be used. The processing conditions described above are also applicable to glucan extraction from fungi in general. The properties of these glucans also will depend on the sources from which they are derived.

According to a first chemical treatment, the whole glucan particles can be treated with an acid to decrease the amount of β(1-6) linkages and thus, change the hydrodynamic properties of said glucans as evidenced by an increase in the viscosity of aqueous solutions of these modified glucans.

A process for preparing an altered whole glucan particles by treating the glucan particles with an acid, for a suitable period of time to alter the β(1-6) linkages can also be used. Acetic acid is preferred, due to its mild acidity, ease of handling, low toxicity, low cost and availability, but other acids may be used. Generally these acids should be mild enough to limit hydrolysis of the β(1-3) linkages. The treatment is carried out under conditions to substantially only affect the β(1-6) linked glucans. In certain embodiments, the acid treatment is carried out with a liquid consisting essentially of acetic acid, or any dilutions thereof (typical diluents can be organic solvents or inorganic acid solutions). The treatment is carried out at a temperature of from about 20° C. to about 100° C. In certain embodiments, the treatment is carried out to such an extent to remove from about 3 to about 20 percent by weight of acid soluble material based on total weight of the whole glucan particles before treatment. In other embodiments, the extent of removal is from about 3 to about 4 percent by weight. Certain compositions formed demonstrate altered hydrodynamic properties and an enhancement in viscosity after treatment.

According to a second chemical treatment, the whole glucan particles are treated with an enzyme or an acid, to change the amount of β(1-3) linkages. For whole glucan particles derived from some yeast strains, enzyme treatment causes a decrease in the viscosity, and for others, it causes an increase in viscosity, but in general, alters the chemical and hydrodynamic properties of the resulting glucans. The treatment is with a β(1-3) glucanase enzyme, such as laminarinase, for altering the β(1-3) linkages to alter the hydrodynamic properties of the whole glucan particles in aqueous suspensions.

The enzyme treatment can be carried out in an aqueous solution having a concentration of glucan of from about 0.1 to about 10.0 grams per liter. Any hydrolytic glucanase enzyme can be used, such as laminarinase, which is effective and readily available. The time of incubation may vary depending on the concentration of whole glucan particles and glucanase enzyme. The β(1-3) linkages are resistant to hydrolysis by mild acids such as acetic acid. Treatment with strong or concentrated acids, such as hydrochloric acid (HCl), sulfuric acid (H₂SO₄) or formic acid, hydrolyzes the β(1-3) linkages thereby reducing the amount of β(1-3) linkages. The acid treatment can be carried out in an aqueous solution having a concentration of glucan from about 0.1 to about 10.0 grams per liter. The time of acid treatment may vary depending upon the concentration of whole glucan particles and acid. Acid hydrolysis can be carried out at a temperature of from about 20° C. to about 100° C. The preferred compositions formed demonstrate altered hydrodynamic properties.

By controlling the incubation time, it is possible to control the chemical and hydrodynamic properties of the resulting product. For example, the product viscosity can be precisely controlled for particular usage, as, for example, with a variety of food products.

A hydrodynamic parameter (K₁) of the final treated product having altered linkages is dependent on the treatment time according to the final formula:
K₁=−0.0021 (time)+0.26
where time is in minutes; and
where time is less than one hour.

The parameter K₁ is directly related (proportional) to the relative viscosity. In the case of aqueous suspensions the relative viscosity is equal to the actual viscosity when the latter is measured in centipoise.

A process for preparing aqueous slurry of a glucan having a predetermined desired viscosity is provided. The slurry comprises glucan at a concentration that is a function of the predetermined desired viscosity according to the following approximate formula:
1/concentration=K₁×(1/log(relative viscosity))+K₂
Where,
K₁=(shape factor)×(hydrodynamic volume); and
K₂=(hydrodynamic volume)/(maximum packing fraction).

The shape factor is an empirically determined value that describes the shape of the glucan matrix in its aqueous environment. The shape factor is a function of the length: width ratio of a particle and can be determined microscopically. The hydrodynamic volume is a measure of the volume a particle occupies when in suspension. This is an important parameter for glucan suspensions in that it indicates the high water holding capacity of glucan matrices. The maximum packing fraction can be described as the highest attainable volume fraction of glucans that can be packed into a unit volume of suspension.

Preparation of Microparticulate β-Glucan Particle

Beta (1,3) glucan starting material can be isolated from yeast cell walls by conventional methods known by those of ordinary skill in the art. The general method for the production of glucan from yeast involves extraction with alkali followed by extraction with acid (Hassid et al., Journal of the American Chemical Society, 63:295-298, 1941). Improved methods for isolating a purified water insoluble beta (1,3) glucan extract are disclosed in U.S. Pat. No. 5,223,491, which is incorporated herein by reference in its entirety. Another method of producing whole glucan particles is disclosed in U.S. Pat. No. 4,992,540, which is incorporated herein by reference in its entirety. Methods for preparing microparticulate β-glucan particles are disclosed in U.S. Pat. No. 5,702,719, the disclosure of which is incorporated herein by reference in its entirety. Microparticulate glucan product can also be obtained with the average particle size of about 1.0 microns or less or about 0.20 microns or less.

Beta glucan particles can be reduced in size by mechanical means such as by, using a blender, microfluidizer, or ball mill, for example. For example, particle size can be reduced using a blender having blunt blades, wherein the glucan mixture is blended for a sufficient amount of time, preferably several minutes, to completely grind the particles to the desired size without overheating the mixture. Another grinding method comprises grinding the glucan mixture in a ball mill with 10 mm stainless steel grinding balls. This latter grinding method is particularly preferred when a particle size of about 0.20 microns or less is desired.

Prior to grinding, the glucan mixture is preferably passed through a series of sieves, each successive sieve having a smaller mesh size than the former, with the final mesh size being about 80. The purpose of sieving the mixture is to separate the much larger and more course glucan particles from smaller particles (the pore size of an 80 mesh sieve is about 0.007 inches or 0.178 mm). The separated larger particles are then ground down as described above and re-sieved to a final mesh size of 80. The process of sieving and grinding is repeated until a final mesh size of 80 is obtained. The sieved particles are combined and ground down further, preferably for at least an hour, until the desired particle size is obtained, preferably about 1.0 micron or less, more preferably about 0.20 microns or less. Periodic samples of the fine grind glucan are taken during the grinding process and measured using a micrometer on a microscope.

Complement Activating Antibodies

Complement activating antibodies (both naturally found or produced by methods known in the art) are antibodies directed to the tumor or tumor antigens that are able to activate one or more members of the complement cascade. In other words, an antibody that activates complement sufficiently to deposit iC3b on the tumor cells is needed. In certain embodiments, the antibodies are IgG subclass I or IgG subclass II.

The present invention discloses the use of NSG with antibodies from essentially any source, including antibodies generated naturally in response to infection, antibodies generated in response to administration of a vaccine, and monoclonal antibodies directly administered as part of a therapy including the use of β-glucan. Any antibody having complement activating features can be used in the methods described herein to enhance beta-glucan on tumorcidal activity. The antibody can also be a naturally occurring antibody found in the subject that is able to activate complement sufficiently to allow deposition of iC3b on the tumor cells. Murine antibodies can be raised against any antigen associated with neoplastic (tumor) cells using techniques known in the art. In this regard, tumor cells express increased numbers of various receptors for molecules that can augment their proliferation, many of which are the products of oncogenes. Thus, a number of monoclonal antibodies have been prepared which are directed against receptors for proteins such as transferring, IL-2, and epidermal growth factor. It suffices to say that any antibody that can selectively label antigen—which is to say any antibody—could have its activity enhanced through concurrent administration with β-glucan. This includes antibodies of the various classes, such as IgA, IgD, IgE, and IgM, as well as antibody fragments such as Fab.

The term “antibody” as used herein refers to immunoglobulin molecules and immunologically active portions of immunoglobulin molecules, i.e., molecules that contain an antigen binding site that specifically binds a tumor antigen. A molecule that specifically binds to tumor is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The term “monoclonal antibody” or “monoclonal antibody composition”, as used herein, refers to a population of antibody molecules that contain only one species of an antigen binding site capable of immunoreacting with a particular epitope of a target tumor A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g. Current Protocols in Immuniology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g. an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

The present invention discloses the use of whole glucan particles with antibodies from essentially any source, including antibodies generated naturally in response to infection, antibodies generated in response to administration of a vaccine, and monoclonal antibodies directly administered as part of a therapy including the use of β-glucan. The majority of humanized mAbs containing the human IgG1 Fc-region have been shown to activate complement, such as Herceptin™ (trastuzumab), Rituxan™ (rituximab), and Erbitux™ (cetuximab) (Spiridon, C. I., et al., Clin. Cancer Res., 8: 1720-1730 (2002), Idusogie, E. E., et al., J. Immunol., 164: 4178-4184 (2000), Cragg, M. S., et al., Blood, 101: 1045-1052 (2003), Herbst, R. S. and Hong, W. K., Semin. Oncol., 29: 18-30 (2002). In certain embodiments the whole glucan particles and antibodies work synergistically.

As illustrative of the inventive concept, β-glucans such as whole glucan particles could be administered to act synergistically with Herceptin™, a monoclonal antibody sold by Genentech for use in immunotherapy of breast cancer. Herceptin™ is a mAb that recognizes the her2 cell surface antigen which is present on 20% of breast cancer cell types. Clinical trials have demonstrated that Herceptin™ is saving lives, but its effectiveness could be significantly enhanced through concurrent administration of β-glucan. NSG therapy along with Herceptin™ therapy could result in a significant increase in the proportion of women responding to Herceptin™ therapy with long lasting remission of their breast cancer. Currently, only 15% of women receiving Herceptin™ therapy show long lasting remission.

Another mAb whose activity is enhanced by whole glucan particles is rituximab, a monoclonal antibody used to treat a type of non-Hodkin's lymphoma (NHL), a cancer of the immune system. Rituxan™, (Rituximab) is effective for patients with low-grade B-cell NHL who have not responded to standard treatments. It targets and destroys white blood cells (B-cells) that have been transformed, resulting in cancerous growth. Rituximab is a genetically engineered version of a mouse antibody that contains both human and mouse components. In the main clinical study of 166 patients with advanced low-grade or slow-growing NHL, which represents about 50% of the 240,000 NHL patients in the United States, tumors shrunk by at least one half in 48% of the patients who completed treatment with rituximab, with 6% having complete remission. Beta-glucan can be expected to significantly increase the effectiveness of this treatment, by enhancing the destruction of antibody-marked tumor cells.

Formulation and Administration

The administration of the whole glucan particles and complement activating antibodies can be administered sequentially, co-administered or in multiple dosing. Further, the order of administration is interchangeable and the antibody can be naturally existing.

Oral formulations suitable for use in the practice of the present invention include capsules, gels, cachets, tablets, effervescent or non-effervescent powders or tablets, powders or granules; as a solution or suspension in aqueous or non-aqueous liquid; or as an oil-in-water liquid emulsion or a water-in-oil emulsion. The compounds of the present invention may also be presented as a bolus, electuary, or paste.

Generally, formulations are prepared by uniformly mixing the active ingredient with liquid carriers or finely divided solid carriers or both, and then if necessary shaping the product A pharmaceutical carrier is selected on the basis of the chosen route of administration and standard pharmaceutical practice. Each carrier must be “acceptable” in the sense of being compatible with the other ingredients of the formulation and not injurious to the subject. This carrier can be a solid or liquid and the type is generally chosen based on the type of administration being used. Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g., PEG, are also preferred carriers.

The formulations for oral administration may comprise a non-toxic, pharmaceutically acceptable, inert carrier such as lactose, starch, sucrose, glucose, methyl cellulose, magnesium stearate, dicalcium phosphate, calcium sulfate, mannitol, sorbitol, cyclodextrin, cyclodextrin derivatives, or the like.

Capsule or tablets can be easily formulated and can be made easy to swallow or chew. Tablets may contain suitable carriers, binders, lubricants, diluents, disintegrating agents, coloring agents, flavoring agents, flow-inducing agents, or melting agents. A tablet may be made by compression or molding, optionally with one or more additional ingredients. Compressed tables may be prepared by compressing the active ingredient in a free flowing form (e.g., powder, granules) optionally mixed with a binder (e.g., gelatin, hydroxypropylmethylcellulose), lubricant, inert diluent, preservative, disintegrant (e.g. sodium starch glycolate, cross-linked carboxymethyl cellulose) surface-active or dispersing agent. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, or the like. Lubricants used in these dosage forms include sodium oleate, sodium stearate, magnesium stearate, sodium benzoate, sodium acetate, sodium chloride, or the like. Disintegrators include, for example, starch, methyl cellulose, agar, bentonite, xanthan gum, or the like. Molded tablets may be made by molding in a suitable machine a mixture of the powdered active ingredient moistened with an inert liquid diluent.

The tablets may optionally be coated or scored and may be formulated so as to provide slow- or controlled-release of the active ingredient Tablets may also optionally be provided with an enteric coating to provide release in parts of the gut other than the stomach.

Exemplary pharmaceutically acceptable carriers and excipients that may be used to formulate oral dosage forms of the present invention are described in U.S. Pat. No. 3,903,297 to Robert, issued Sep. 2, 1975, incorporated by reference herein in its entirety. Techniques and compositions for making dosage forms useful in the present invention are described in the following references: 7 Modem Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976).

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Extemporaneous injections solutions and suspensions may be prepared from sterile powders, granules and tablets of the kind previously described. Parenteral and intravenous forms may also include minerals and other materials to make them compatible with the type of injection or delivery system chosen.

All publications cited are incorporated by reference in their entirety. The present invention is now illustrated by the following Exemplification, which is not intended to be limiting in any way.

### EXEMPLIFICATION

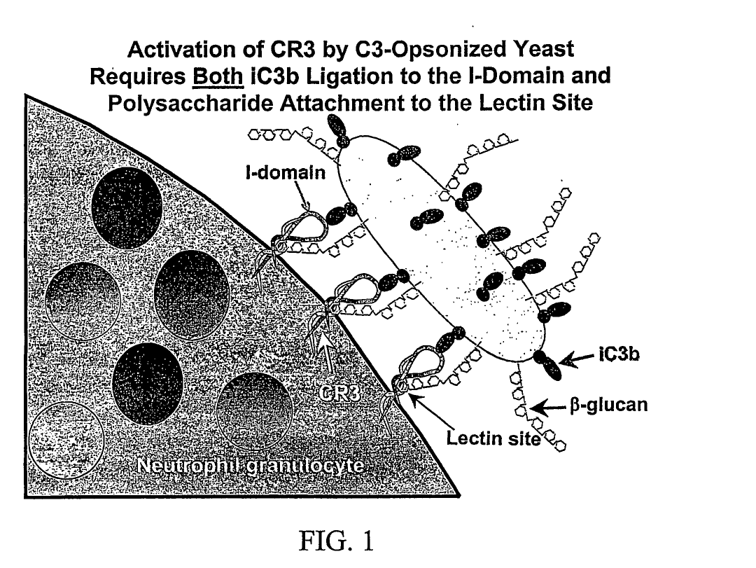

CR3 plays a very important role in the antitumor activity of whole glucan particles. The role of CR3 in mediating the response to whole glucan particles was shown by research into the mechanisms of neutrophil phagocytosis of iC3b-opsonized yeast. When complement C3b has attached itself to a surface, it may be clipped by a serum protein to produce a smaller fragment, iC3b. While iC3b has been “inactivated” and cannot function to form a membrane attack complex, it remains attached to the surface where it serves to attract neutrophils and macrophages which can phagocytize or otherwise destroy the marked (“opsonized”) cell. On the surface of neutrophils and macrophages are complement receptors (CR3) that bind to iC3b. The process by which yeast is eliminated by the immune system is illustrated in FIG. 1.

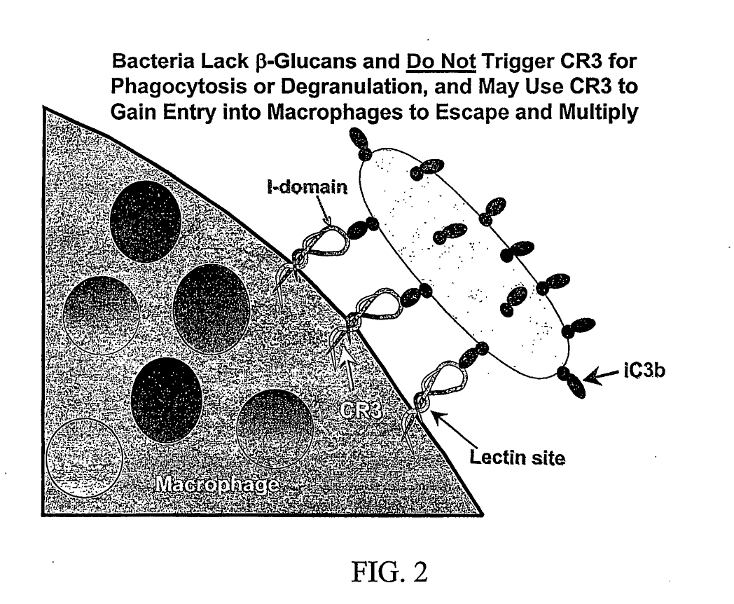

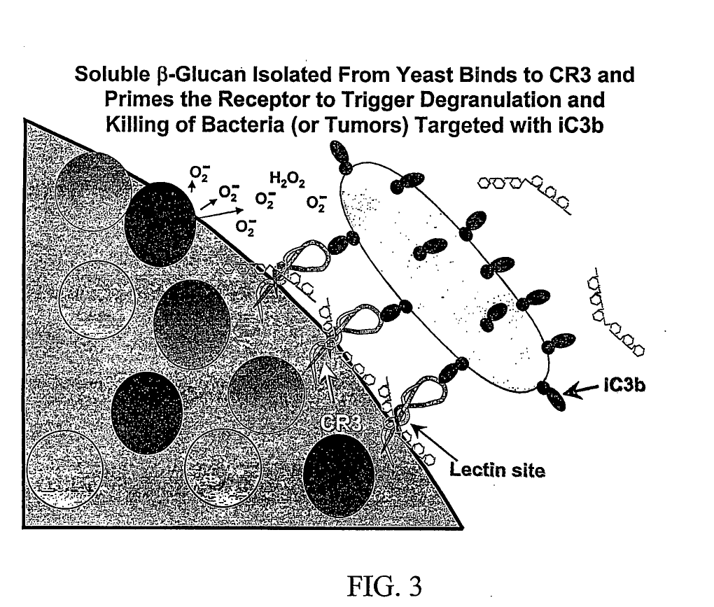

Stimulation of CR3-dependent phagocytosis or degranulation requires the simultaneous ligation of two distinct sites within CR3; one specific for iC3b and a second site specific for yeast cell wall whole glucan particles. As illustrated in FIG. 2, because they lack cell-surface CR3-binding whole glucan particles, bacterial opsonized with iC3b are bound to neutrophils via CR3 but do not stimulate phagocytosis or degranulation. However, as illustrated in FIG. 3, addition of whole glucan particles that are degraded and brought to the tumor site can bind to the lectin site of CR3 to activate immune cells bearing the receptor to trigger degranulation and or phagocytosis of the foreign material. Soluble zymosan-derived polysaccharides rich in mannans and β-glucans have been shown to bind to CR3 with high affinity, inducing a primed receptor state.

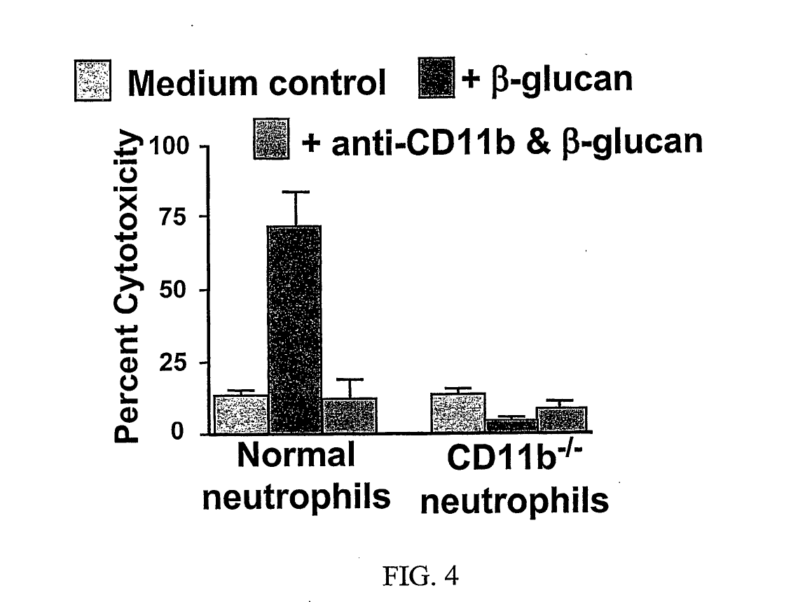

The effect of whole glucan particles priming of murine neutrophil CR3 on subsequent cytotoxic triggering by iC3b-opsonized breast tumor cells is shown in FIG. 4. When normal neutrophils are used, addition of β-glucan creates a high level of cytotoxicity towards iC3b-opsonized breast tumor cells. This activity disappears, however, when antibody to the CD11b (murineCR3 equivalent) is added, interfering with the receptor's ability to bind to iC3b. On the right side of the graph, it is demonstrated that neutrophils from CD11b-deficient mice are unable to mediate cytotoxicity of iC3b-opsonized mice even when stimulated with β-glucan, again demonstrating the crucial importance of this receptor. Adding antibody against CD11b had little effect on CD11b-deficient neutrophils, as expected.

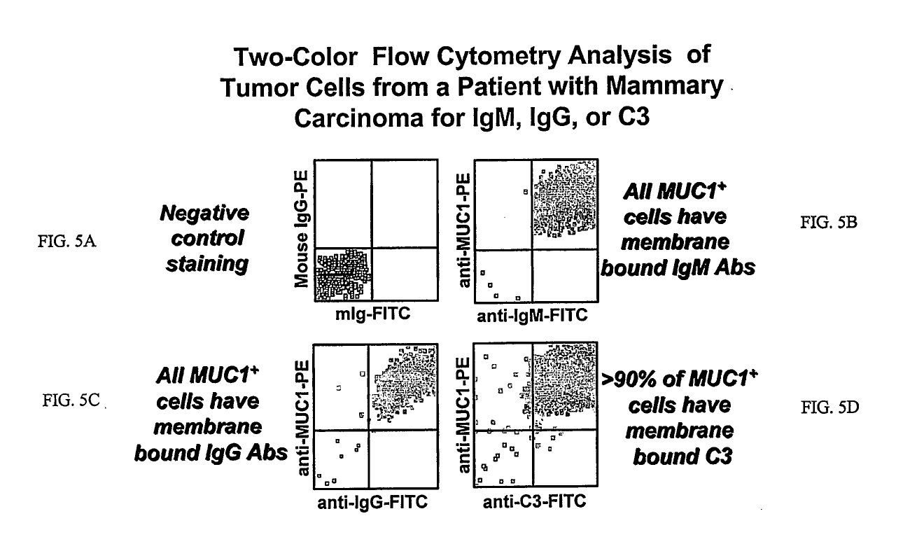

FIG. 5 illustrates that tumor cells are coated with IgG, IgM, and C3. This is interesting because it shows that a weak adaptive immune response is occurring against these tumor cells, and that if this labeling could be used to trigger a cytotoxic response tumor growth could be inhibited or eliminated. Two-color flow cytometry was used to distinguish breast tumor cells from normal breast epithelial cells, and then to show that most tumor cells bear IgG, IgM, and C3. Single cell suspensions of freshly excised tumors from two patients were analyzed. FIG. 5 shows the results obtained with the tumor from one of the patients. Malignant cells were distinguished from normal breast epithelial cells by staining with biotinylated anti-mucin (MUC1)-biotin mAb BrE-3 followed by streptavidin phycoerythrin. Two-color analysis was then used to determine the presence of IgG, IgM, and C3 on malignant MUC1-positive cells by double staining with antibodies coupled to fluorescein isothiocyanate (FITC). As was seen, most of the MUC1-positive cells bore IgG, IgM, and C3. Only a small proportion of the MUC1-positive cells appeared to be negative for opsonization with C3. Little, if any, C3 or Ig staining was detected on MUC1-negative cells, which represent normal breast epithelial cells.

Whole glucan particles, on the other hand, binds to CR3, stimulating neutrophil degranulation and stimulating macrophages to secrete several cytokines that promote a Th1-type T cell response and long-lasting immunity to tumors or microorganisms. Particular β-glucan can also prime CR3 for cytotoxicity in vitro or in vivo.

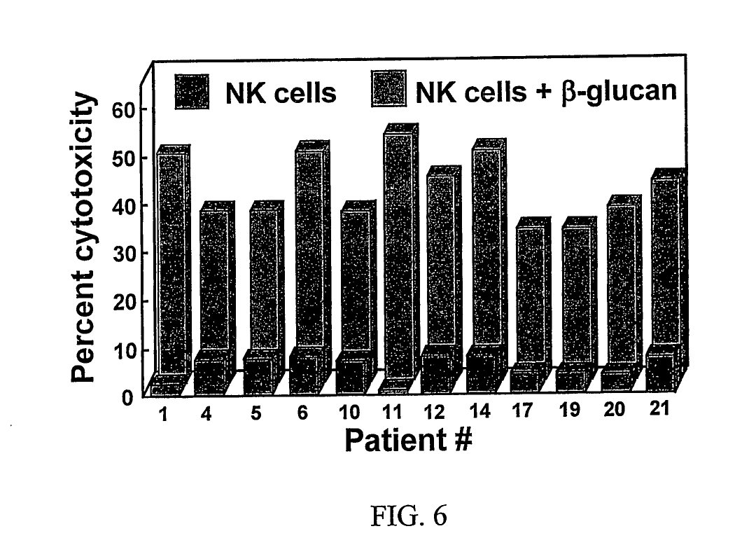

NK cells are an important component of the innate immune system, and can kill tumor cells by stimulating apoptosis through the Fas ligand or through formation of a MAC complex and insertion of apoptosis-inducing enzymes. NK cells complement the activity of macrophages by targeting cells that have lost their MHC proteins through tumor or viral action. Target cell-bound C3 is also required for NK cell CR3-dependent cytotoxicity. As shown in FIG. 6, tests were conducted with freshly excised tumors from 12 patients to determine whether the tumor cells bore sufficient amounts of C3 for recognition and cytotoxicity by NK cells bearing soluble zymosan polysaccharide (SZP)-primed CR3. Fresh and viable tumor cell suspensions were labeled with ⁵¹Cr and tested for susceptibility to the cytotoxicity of NK cells isolated from a normal, unrelated donor during a 4 hour incubation period at 37° C. It also demonstrates the powerful enhancement of NK cell activity by addition of β-glucan in the form of SZP. Although no significant cytotoxicity was observed with unstimulated NK cells, priming the cells with 2.0 μg/ml of SZP resulted in 32 to 54% cytotoxicity. The presence of C3-negative normal breast epithelial cells that were surgically removed along with the tumor probably prevented higher levels of cytotoxicity from being shown.

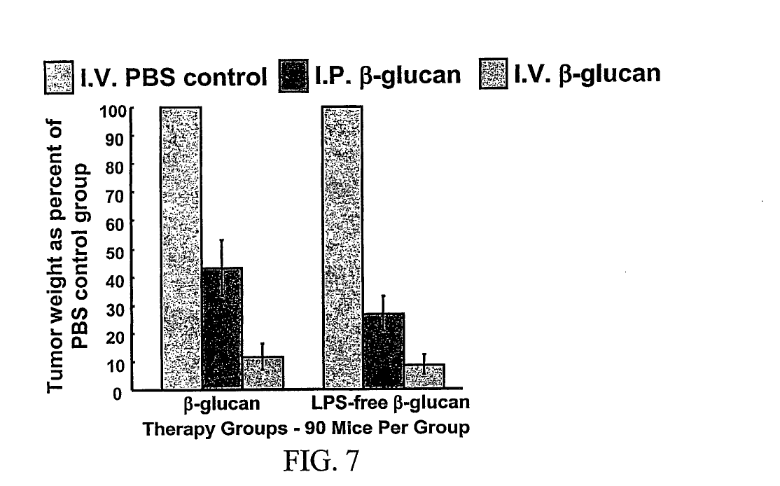

The efficacy of β-glucan therapy is demonstrated in FIG. 7, which shows the results of β-glucan therapy of Ptas64 mammary carcinoma in Balb/c mice. SZPₘ (soluble zymosan polysaccharide rich in β-mannans) was used as the β-glucan source in this experiment. Ptas64 mammary carcinoma was implanted in Balb/c mice. For each of six experiments, two groups of 6 mice were given daily i.p. or i.v. injections of 200 μg of SZPₘ. A control group of 6 mice received daily i.v. phosphate buffered saline (PBS). Three experiments with 30 mice were carried out with SZPₘ, and then the same experiment was done 3 more times with another 90 mice using LPS-free SZPₘ. For each experiment, the average tumor weight for the therapy groups was determined and compared to the average weight of tumors removed from the PBS control group. Each bar in FIG. 7 represents the mean±SD for each therapy group. As can be seen, tumor weight was dramatically reduced to 40 and 10% for i.p. and i.v. administration of β-glucan, respectively. Furthermore, the experiments with LPS-free SZPₘ demonstrate that this activity is not due to LPS, which is a well known immunostimulant.

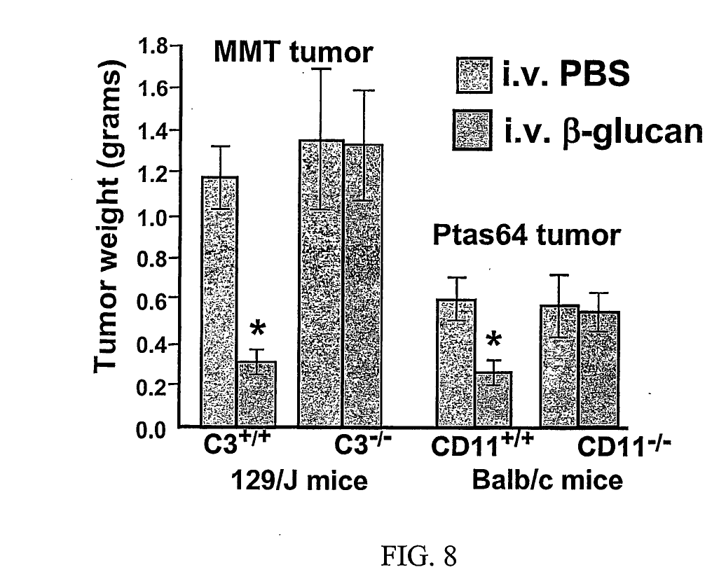

FIG. 8 demonstrates that β-glucan therapy requires both C3 on tumor cells and CR3 on leukocytes. The requirement for C3 in β-glucan therapy was confirmed in experiments with C3-deficient 129/J mice implanted with the M mammary carcinoma. Twelve normal (C3+/+) and 12 C3-deficient (C3−/−) 129/J mice were implanted with the MMT mammary carcinoma tumor cell line and palpable tumors were allowed to form before beginning daily i.v. therapy of groups of 6 mice each with PBS or β-glucan. A similar protocol was used with normal (CD+/+) and CR3-deficient (CD11b-knockout; Cd11b−/−) BALB/c mice implanted with Ptas64 mammary tumors. Beta-glucan therapy of normal 129/J mice resulted in a 79% tumor reduction, as shown in the figure, similar to that of normal BALB/c mice. Flow cytometry of the tumors showed an abundant deposition of C3 on >80% of cells. By contrast, in C3-deficient 129/J mice, there was no significant tumor reduction and no C3 present on the tumors. The relative amount of IgG present on the tumors, as demonstrated by staining, did not differ between normal and C3-deficient mice.

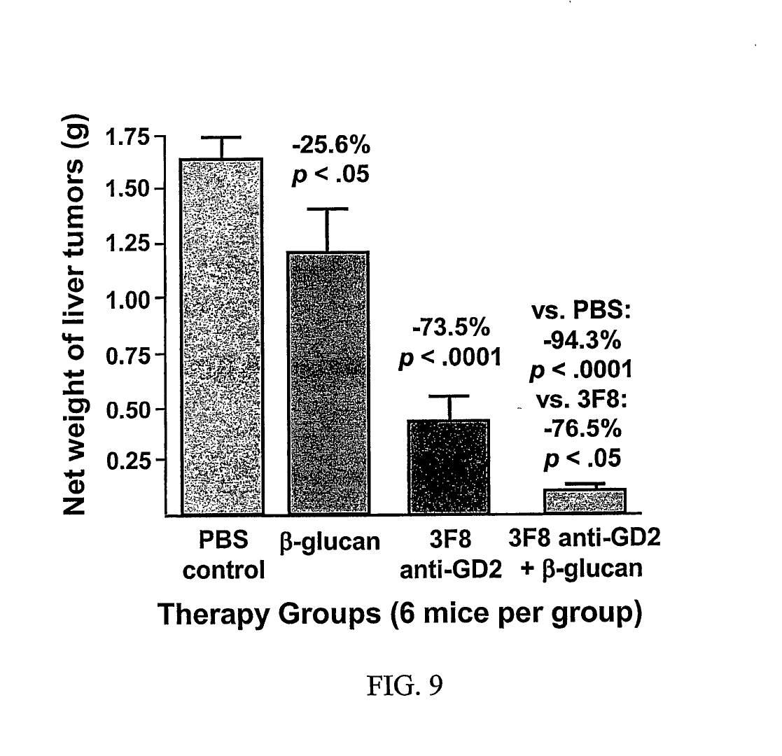

The next step was to demonstrate that β-glucan could be used to enhance mAb therapy. The results of these experiments are shown in FIG. 9, which shows the enhancement of anti-tumor mAb therapy of hepatic ELM lymphoma with β-glucan. The EL-4 lymphoma was tested for a response to glucan therapy because, unlike other tumors, the syngeneic host (C57BL/6) did not express natural Abs that opsonized the tumor cells with C3. Mice were injected with the EL-4 cells i.v., a protocol known to result in liver metastases. Ten days later, the mice were given daily i.v. doses of β-glucan alone, 3F8 mAb to GD2 ganglioside (a prominent tumor antigen of EL-4 cells), or β-glucan plus 3F8 mAb. The 3F8 mAb is IgG3 and is a potent activator of complement and also mediates ADCC. As other had shown, 3F8 alone caused a significant reduction (73.5%) in EL-4 liver tumors. As expected, the β-glucan had little effect on its own since the tumors bore little or no iC3b. However, the combination of β-glucan with 3F8 produced a significantly greater reduction in liver tumor compared to 3F8 alone.

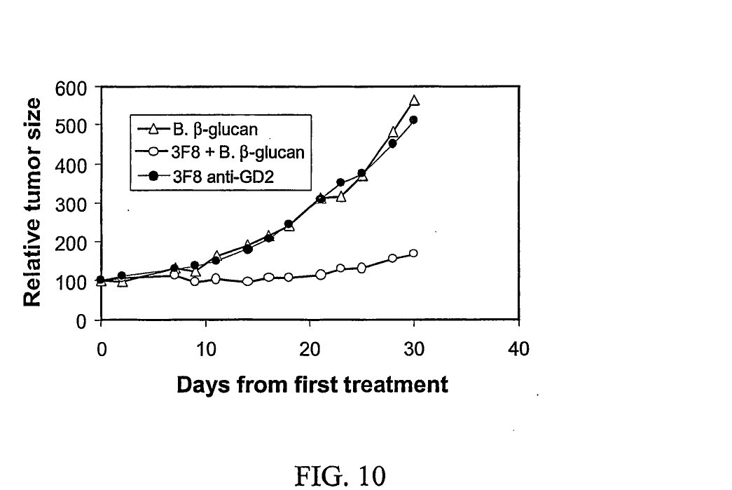

FIG. 10 demonstrates that β-glucan from barley (which produces the unusual (1,3), (1,4)-β-D-glucan) can also enhance the antitumor activity of mAbs. Neuroblastoma cells were xenografted subcutaneously in athymic BALB/c mice. Treatment started in groups of 5 mice, 2 weeks after tumor implantation when visible tumors reached 0.7-0.8 cm in diameter. The β-glucan groups were treated with 400 μg daily by gavage for a total of 21-29 days. Monoclonal antibodies (3F8) were administered i.v. at a dose of 200 μg twice weekly. Tumor size was measured from the first day of treatment, and the product of the largest diameter was expressed as a percentage of the size on day zero. As can be seen, neither β-glucan or mAb alone showed much effect However, when the two are combined, tumor growth is clearly suppressed.

TABLE 1
Sugar and CR3 specificity of neutrophil fluorescence staining by
FITC-labeled polysaccharides

| | Polysaccharide-FITC | Specific Fluorescence Mean | | |
|---|---|---|---|---|
| | Concentration Required | Channel at Saturation (Total- | % inhibition of Specific | |
| | for Maximum Specific | Nonspecific Fluorescence | Fluorescence by | |
| Type of | Fluorescence Staining | Obtained in the Presence of | | MN-41 |
| FITC-Labeled | (Saturation of Receptor | Excess of Unlabeled | SZP | anti-CR3 |
| Polysaccharide | Binding Sites) (μg/ml) | Polysaccharide) | (50 μg/ml) | (50 μg/ml) |
| Dextran-FITC | No specific staining | 0 | ND | ND |
| α-Mannan-FITC | No specific staining | 0 | ND | ND |
| Barley β-glucan- | 10 | 12.8 | 92.3 | 94.5 |
| FITC | | | | |
| CM β-glucan-FITC | 5 | 22.2 | 87.8 | 78.9 |
| Laminarin-FITC | 10 | 38.2 | 91.5 | 89.9 |
| Lentinan-FITC | 5 | 21.5 | 96.3 | 81.0 |
| MP β-glucan-FITC | 2 | 50.9 | 100 | 96.1 |
| SZP-FITC | 2 | 175 | 99.8 | 77.4 |

Table 1 shows the sugar specificity of β-glucan from various and also shows the results of flow cytometry with various pure β-glucan-FITC preparations. No specific staining was obtained with dextran-FITC or α-mannan-FITC. Even though each polysaccharide-FITC preparation produced a lower intensity of neutrophil staining than did SZP-FITC, the fluorescence of each polysaccharide-FITC was similarly inhibited by excess unlabeled homologous β-glucan, unlabeled SZP, or anti-CR3. Comparison of polysaccharide concentrations required for maximum staining suggested that SZP or MP β-glucan (a soluble β-glucan obtained from Molecular Probes) had the highest affinity, since saturation with either required 2 μg hexose/ml. Fluorescence intensity values obtained with individual polysaccharide-FITC conjugates cannot be compared, since the molar ratio of FITC to polysaccharide is likely to differ and cannot be readily calculated.

Comparison of the concentrations of β-glucans required for 50% inhibition of SZP-FITC staining suggested that CR3 had a somewhat higher affinity for SZP than for β-glucan. Neutrophils were incubated at 4° C. with graded concentrations of β-glucans (SZP, laminarin, MP β-glucan, barley β-glucan, and lentinan), α-mannan or dextran for 15 minutes and then stained by addition of 1.0 mg/ml of SZP-FITC and incubated for an additional 15 minutes at 4° C. Percentage inhibition was then compared with polysaccharide concentration. Whereas 50% of inhibition of SZP-FITC staining required 0.2 μg of hexose/ml of unlabeled SZP, 50% inhibition of SZP-FITC by unlabeled β-glucans required 5 mg of hexose/ml (MP β-glucan or laminarin) to 75 ug hexose/ml (lentinan). Similar results were obtained when the same unlabeled polysaccharides were examined for inhibition of laminarin β-glucan-FITC staining. The rank order for inhibiting activity for both experiments was SZP >laminarin >MP β-glucan. However, CM β-glucan (carboxymethyl β-glucan from yeast), barley β-glucan, and lentinan inhibited laminarin-FITC staining more efficiently than they inhibited SZP-FITC staining. Overall, these results demonstrate that barley β-glucan has a relatively low affinity for CR3 than did soluble yeast MP β-glucan or SZP.

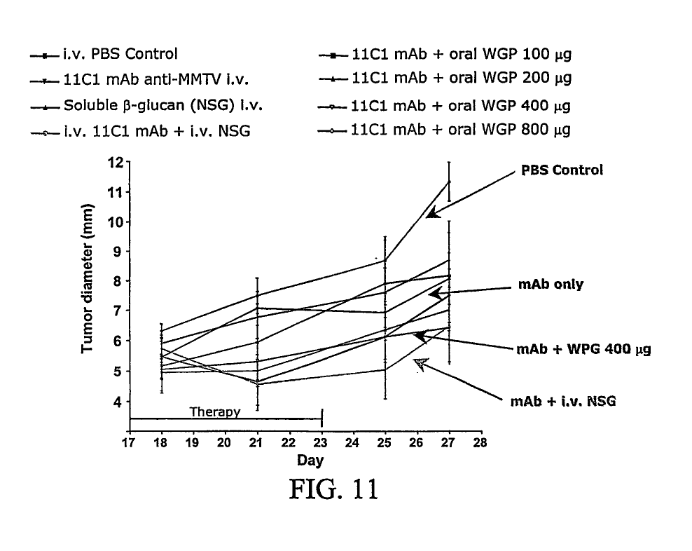

Balb/c mammary carcinoma was then treated with i.v. mAb and oral WGP at various concentrations or i.v. NSG to determine the relative in vivo activity of these combinations, as shown in FIG. 11. This demonstrated the effectiveness of both the NSG and WPG forms of β-glucan, as well as the oral and intravenous administration routes. The NSG and WPG forms demonstrated comparable effectiveness, with 400 μg of oral WGP providing the strongest activity relative to other doses of WGP. Visual inspection of mice again confirmed these results. Mice in the control group receiving i.v. mAb 11C1 only showed tumors of about 9 mm in diameter. In mice receiving i.v. mAb 11C1 plus i.v. soluble β-glucan (NSG), 4 out of 5 mice showed no visible tumor. In mice receiving i.v. mAb 11C1 plus oral WPG at 200 μg/day, tumors were 20% the size of controls, and 2 out of 5 mice had only barely visible tumors.

The present invention discloses the use of particulate β-glucan synergistically with antibodies from essentially any source, including antibodies generated naturally in response to infection, antibodies generated in response to administration of a vaccine, and monoclonal antibodies directly administered as part of a therapy including the use of β-glucan.

In certain embodiments, an antibody that activates complement sufficiently to deposit iC3b on the tumor cells in needed. In most cases antibodies are evaluated for their ability to mediate direct killing of the cancer cells by complement alone. Except with Rituxan, most therapeutic antibodies activate complement but the complement is unable to kill the tumor cells. This is irrelevant to glucan therapy. It is not required that the complement kill the tumor, what is needed is having the complement target the tumor with surface bound iC3b. The deposition of iC3b on the tumor requires considerably less complement activation than is required to kill a tumor with complement. Hence the presence of complement inhibitors on tumors, that prevent sufficient complement activation for direct killing by complement, do allow enough complement activation to tag the tumors with iC3b.

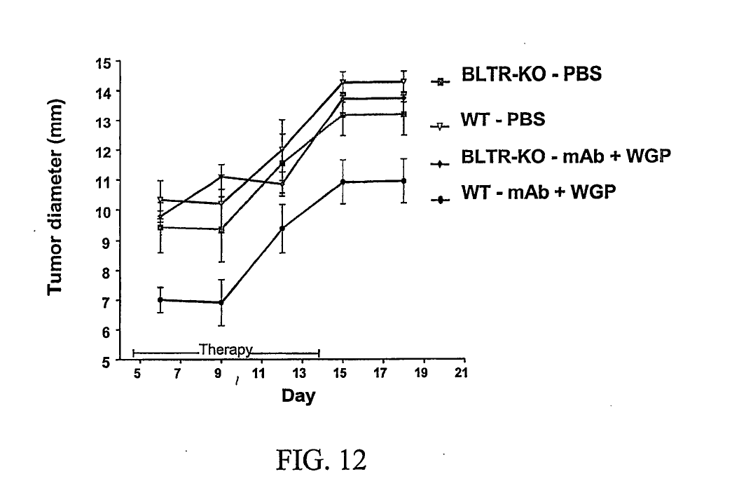

FIG. 12 illustrates how leukocyte recruitment via leukotriene B₄ receptors (BLTR) is involved in the tumoricidal activity of combined oral WGP and mAb therapy. This graph demonstrates that monocytes and neutrophils are involved in the antitumor response, in addition to NK cells. LTB4 is a chemoattractant, produced predominantly by neutrophils and macrophages. It is involved in a number of events, including: stimulation of leukocyte migration from the bloodstream; activation of neutrophils; inflammatory pain; host defense against infection; increased interleukin production and transcription. Since the presence of LTB4 is important for the antitumor activity of β-glucan, it is clear that macrophages and neutrophils are involved in the immune response in addition to NK cells.

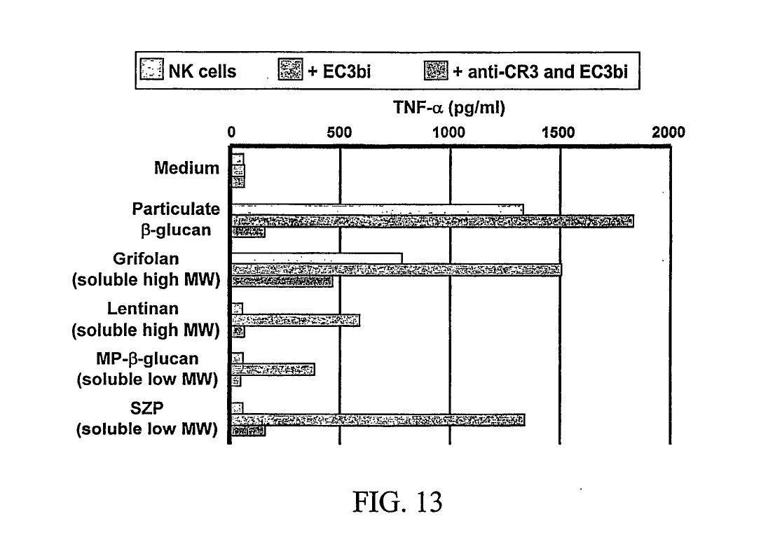

The function of NK cells in mediating host defense includes both direct cytotoxicity of tumor cells and the secretion of cytokines such as TNF-α and IFN-γ that can potentially regulate immune responses and recruit tumoricidal macrophages. Although direct cytotoxicity of tumors by NK cells has been shown to be mediated by the activation of CR3, additional studies have shown that this same CR3 activation event might also trigger cytokine secretion. Experiments were conducted to confirm this point, the results of which are shown in FIG. 13. This figure shows β-glucan CR3-dependent stimulation of TNF-α secretion by NK cells. Human NK cells were cultured with either particulate yeast β-glucan or soluble CR3-binding polysaccharides for 18 hours at 37° C. Culture supernatants were then analyzed for TNF-α by ELISA. Particulate yeast β-glucan (2 μg/ml) and grifolan (500 kDa soluble β-glucan from Grifola Frondosa, 2 μg/ml) are able to bind and crosslink the lectin sites of surface CR3 molecules, causing cellular activation and the secretion of both TNF-α and IL 6 (not shown). By contrast, the small (20 kDa) soluble yeast β-glucan (MP β-glucan; 2.0 μg/ml) and SZP (soluble zymosan polysaccharide preparation containing β-oligomannan and/or β-glucan; 2.0 μg/ml) bind only to individual CR3 molecules and do not trigger cytokine release in the absence of target cells. As with NK cell CR3-dependent cytotoxicity, binding of small β-glucans to CR3 resulted in receptor priming for subsequent cytokine release triggered by ligation to an iC3b-opsonized target cell (sheep erythrocytes opsonized with iC3b—“+EC3b”). The EC3bi targets did not trigger NK cell cytokine release in the absence of such polysaccharide priming, as shown in the medium control. After polysaccharide priming of CR3, ligation to an iC3b-target cell resulted in secretion of TNF-α, IFN-γ, IFN-α, and IL-6. Addition of 5 mg/ml of an anti-CD11b mAb (OKM1) blocked the secretion of all four cytokines from NK cells. Anti-CR3 blocks both β-glucan binding to CR3, as well as the binding of primed CR3 to iC3b on the EC3bi target cells.

The results shown in FIG. 13 show that NK cell secretion of cytokines occurred in parallel to CR3 activation for cytotoxicity. Particulate β-glucan, that triggers a vigorous CR3-dependent neutrophil superoxide burst, likewise triggered NK cell CR3-dependent release of cytokines. Cytokine secretion did not occur with the initial CR3 priming step that occurs with the binding of small soluble β-glucans to CR3, and occurred only secondarily with the CR3 activation step triggered by cross-linking of the β-glucan primed CR3 to an iC3b-opsonized target cell. Incubation of NK cells with EC3bi in medium alone, that does not stimulate NK cell lysis of the EC3bi, also did not trigger cytokine secretion. However, when EC3bi was added after priming of NK cell CR3 with soluble (or particulate) β-glucan, then the secretion of TNF-α, IFN-α, IFN-γ, and IL-6 was detected by ELISA. Such cytokine release was CR3-dependent because it was blocked when an anti-CD121b mAb was added at the same time as the target EC3bi.

This data suggests a further explanation for the successful use of β-glucans in cancer immunotherapy. In addition to the cytotoxicity triggered when a β-glucan primed NK cell enters a tumor opsonized with iC3b, the same localized cytotoxicity stimulated by the iC3b-opsonized tumor cells would be accompanied by a local, rather than systemic, release of cytokines.

### Example 2

### Materials and Methods

Antibodies and Other Reagents.

The hybridoma producing 11C1 IgG2a anti-MMTV (Raychaudhuri, S., et. al., J. Immunol, 137: 1743-1749 (1986)) was generously provided by Dr. Hiroshi Fugi (Department of Molecular Immunology, Roswell Park Cancer Institute, Buffalo, N.Y.). The 3F8 IgG3 anti-G_{D2} ganglioside mAb (Saito, M., Yu, R. K., and Cheung, N.-K. V., Biochem. Biophys. Res. Commun., 127:1-7, 1985; Cheung, N.-K. V., J. Nucl. Med., 28: 1577-1583 (1987), purified and in sterile citrate-buffered saline, was generously provided by Dr. Nai-Kong V. Cheung (Memorial Sloan-Kettering Cancer Center, New York, N.Y.). Purified 14.G2a IgG2a anti-G_{D2} mAb (Hank, J. A., et al., Cancer Res., 50: 5234-5239, 1990; Uttenreuther-Fischer, M. M., Huang et al., Cancer Immunol. Immunother., 41: 29-36, 1995.), as well as the hybridoma, was generously provided by Dr. Ralph A. Reisfeld (Research Institute of Scripps Clinic, La Jolla, Calif.). The BCP8 hybridoma producing IgG2b anti-human MUC1 mAb (King, P. X., et al., Cancer Res., 52: 2310-2317 (1992) was kindly provided by Dr. Ian F. C. McKenzie (Austin Research Institute, Heidleberg, Australia). The hybridoma producing the rat anti-mouse granulocyte mAb RB6-8C5 (Ly-6G; anti-Gr-1) (Hestdal, K., et al., J. Immunol, 147: 22-28, (1991) was kindly provided by Dr. Emil Unanue (Washington. University School of Medicine, St. Louis, Mo.). The B5 hybridoma secreting mouse IgG2a mAb specific for the human high molecular weight melanoma antigen was obtained from the ATCC (Manassas, VA) and the isolated IgG was used as a “non-specific” mAb control for mouse tumor therapy protocols. Each hybridoma was adapted to grow in 1-2% FCS and BD Hybridoma medium, and then grown in bioreactor flasks (BD Biosciences, San Jose, Calif.) to generate a spent medium rich in mAb that was subsequently purified using sequential steps of ammonium sulfate precipitation, Mono-Q FPLC chromatography, and Mono-S FPLC chromatography. Purified mAbs were sterilized by ultrafiltration and any detectable LPS was removed by extraction with Triton X-114 (Aida, Y. and Pabst, M. J., et al., J. Immunol. Methods, 132: 191-195 (1990)).

Goat anti-mouse antibodies to IgM, IgG, and C3 labeled with fluorescein isothiocyanate (FITC) were purchased from ICN Biomedicals/Cappell (Aurora, Calif.) and used for analysis of Ig and C3 opsonization of tumor cell suspensions using flow cytometry (BD FACScan, BD Biosciences Immunocytometry Systems, San Jose, Calif.). Anti-mouse CD45-PerCP-Cy5.5, anti-mouse CD80-FITC, and anti-Gr-1-PE, anti-mouse CD11c-FITC, as well as appropriately labeled isotype controls, were purchased from BD Biosciences Pharmingen. Rat anti-mouse F4/80-FITC and an isotype control were obtained from Caltag Laboratories (Burlingame, Calif.). Therapeutic β-glucans.

A preparation of whole glucan particles were obtained from Biopolymer Engineering, Eagan Minn. Barley glucan can be obtained from any commercial source such as Sigma Chemical Co., and processed to produced soluble glucan using methods known in the art, such as the methods disclosed in Xia, Y., et al., J. Immunol., 162: 2281-2290 (1999) and Thornton, B. P., et al., J. Immunol., 156: 1235-1246(1996).

Mice and Tumor Models.

Normal BALB/c and C57B1/6 mice were purchased from the Jackson Laboratory (Bar Harbor, Me.) or NCI-Frederick (Frederick, Md.). Heterozygous C3-deficient (C3^{+/-}) mice (31) were purchased from The Jackson Laboratory and used to establish a breeding colony from which were derived both homozygous deficient (C3^{-/-}) and their wild-type (C3^{+/+}) C57BV/6 littermates. A breeding colony of C57B1/6 CR3-deficient (CD11b^{-/-}) mice (Coxon, A., Rieu, et al., Inmmunity, 5: 653-666, 1996) and their wild-type (CD11b^{+/+}) C57BV/6 littermates was obtained from Dr. Tanya Mayadas-Norton (Brigham & Women's Hospital and Harvard Medical School, Boston, Mass.). The phenotypes of the C3^{-/-} and CR3^{-/-} mice and their littermates were confirmed by assays for serum C3 using quantitative radial immunodiffusion and for blood neutrophil CD11b expression using immunofluorescence staining and flow cytometry analysis, respectively.

The BALB/c mammary carcinoma known as Ptas64 (or 64PT) was obtained from Dr. Wei-Zen Wei (Karmonos Cancer Center and Wayne State University, Detroit, Mich.). This tumor line expresses a MMTV (murine mammary tumor virus) membrane antigen detectable with the 11C1 mAb. Previous studies showed that normal BALB/c serum contained naturally-occurring antibodies reactive with Ptas64 that opsonized the tumor cells growing in vivo with IgM, IgG, and C3, and that additional i.v. injections of 11C1 mAb produced increased surface uptake of IgG and C3 (Yan, J., et al., J. Immunol, 163: 3045-3052 (1999). Four groups of mice were injected s.c. with 0.5 to 1.0×10⁶ cells in a mammary fat pad and a tumor was allowed to form over a 7 to 10 day period. When tumor diameters reached 3-4 mm as measured by calipers as an average across the tumor length and width, therapy was initiated. Tumor diameter was measured every 3rd day, and mice were sacrificed when tumor diameters reached 15 mm.

The C57B1/6 lymphoma EL-4, that highly expresses membrane GD2 ganglioside, was provided by Dr. Nai-Kong V. Cheung. Normal C57B1/6 mice were injected with 3×10⁵ EL-4 cells i.v. to generate liver tumors (Zhang, H., et al., Cancer Res., 58: 2844-2849 (1998)). After 2 weeks of therapy, the mice were sacrificed and their livers were removed and weighed in comparison to the livers of a group of normal tumor-free mice. The net weight of liver tumors was calculated by subtracting the weight of a normal liver (1.0 g) from the weights of the livers from tumor-bearing mice. A similar liver tumor model was carried out using the C57B1/6 lymphoma RMA-S that similarly expresses G_{D2} ganglioside but is defective in peptide loading of SMC class I (kindly provided by Dr. Olivera J. Finn, Pittsburgh Cancer Institute, Pittsburgh, Pa.) in combination with 14.G2a mAb to G_{D2} ganglioside (100 μg, given i.v. every 3^{rd} day). Therapy was continued for a period of 3 weeks and the mice were then observed for long-term tumor-free survival.

RMA-S cells transfected with human MUC1 were also provided by Dr. Finn (Soares, M. M., et al., J. Immunol., 166: 6555-6563 (2001)), and 1×10⁶ cell were implanted s.c. in C57B1/6 mice in or near a mammary fat pad. After 8-10 days when tumors of 3-4 mm appeared, therapy was initiated with either 14.G2a anti-G_{D2} or BCP8 anti-MUC1 mAb, with or without β-glucan Therapy was continued for 2 or 3 weeks (as indicated), with tumor measurements made as before, and mice were sacrificed if tumors reached 15 mm in diameter. Mice were observed for tumor-free survival over a total period 90-120 days.

Lewis lung carcinoma cells (LL/2, CRL-1642) originally derived from C57B1/6 mice were obtained from the ATCC and transfected with a plasmid containing cDNA for human MUC1 that was provided by Dr. Olivera Finn (Soares, M. M., et al., J. Immunol, 166: 6555-6563, 2001.). A LL/2 line expressing a uniformly high surface density of MUC1 was selected by FACS sorting of cells stained with BCP8—FITC mAb (MoFlo High Speed Cell Sorter, Dako-Cytomation, Fort Collins, Colo.). A further selection was made by passaging the cell line two times in C57B1/6 mice given the cells sc. A tumor line was selected that both expressed uniformly high levels of surface MUC1 and was capable of generating s.c. tumors in C57B1/6 injected with as few as 5×10⁵ cells. Therapy of mice bearing these s.c. tumors was initiated after 7 days when tumors were only 1-2 mm in diameter. Therapy was given for 3 weeks with measurement of tumor diameters every 3^{rd} day, and mice were sacrificed when tumors reached 15 mm diameter. Mice were observed over a total period of 90 days for tumor-free survival.

Graphing and Statistical Analysis of Data.

All data from mouse tumor therapy protocols was entered into Prism 3.0 (Graph Pad Software, San Diego, Calif.) to generate graphs of tumor regression or survival. Student's T test was next employed within Prism 3.0 to determine the significance of different data sets.

Results

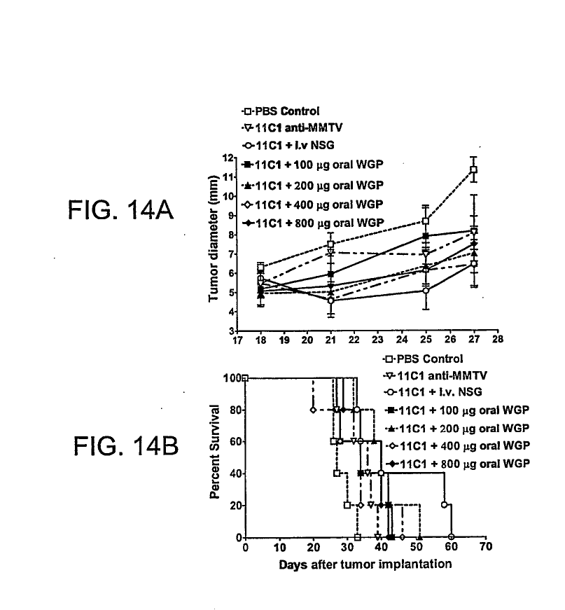

Orally administered yeast whole β-glucan particles (WGP) enhance tumor regression and survival in a similar manner as i.v. yeast β-glucan. As described in Materials and Methods, groups of BALB/c mice were implanted with Ptas64 mammary carcinoma and after 7 days to allow tumor formation were treated for 2 weeks with i.v. 11C1 anti-MMTV with or without simultaneous i.v. NSG yeast β-glucan or oral WGP. The optimal dose of the oral WGP appeared to be 200 to 400 μg per day. Mean values±SD are shown are shown in FIG. 14.

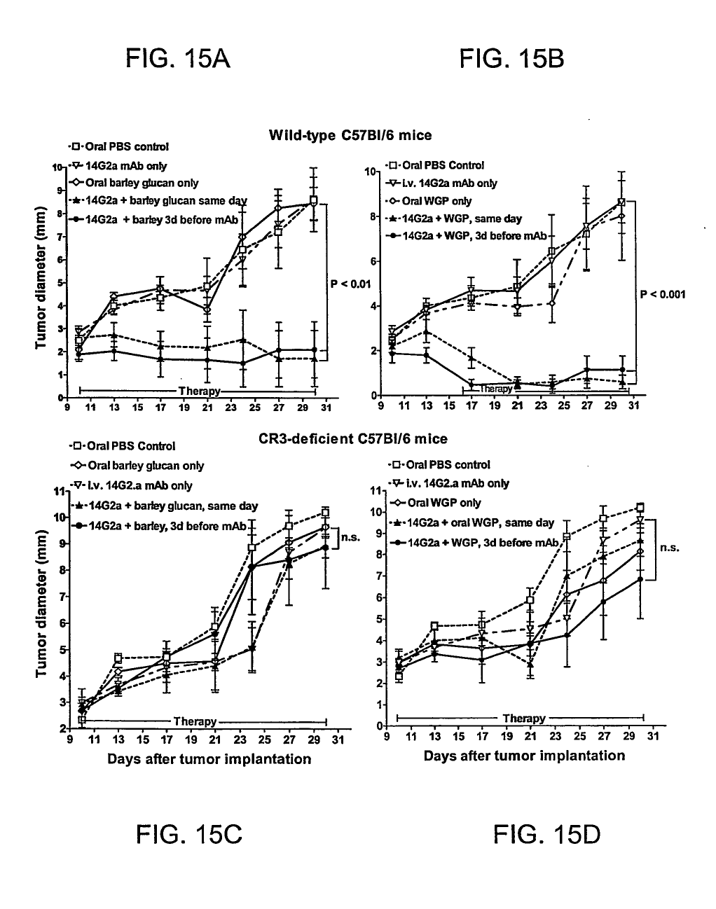

Tumor regression with orally administered soluble barley or particulate yeast β-glucan requires leukocyte CR3. As described in Material and Methods, groups of wild-type or CR3-deficient C57B1/6 mice were implanted s.c. with RMA-S-MUC1 and after 10 days to allow tumor formation were treated with i.v. 14.G2a anti-GD2 ganglioside with or without simultaneous oral barley or yeast β-glucan (WGP). Some groups of mice were given the oral β-glucan on the same day as the i.v. mAb (day 10), whereas other groups of mice were given the oral β-glucan 3 days before the mAb (day 7). Mean values±SD are shown in FIG. 15.

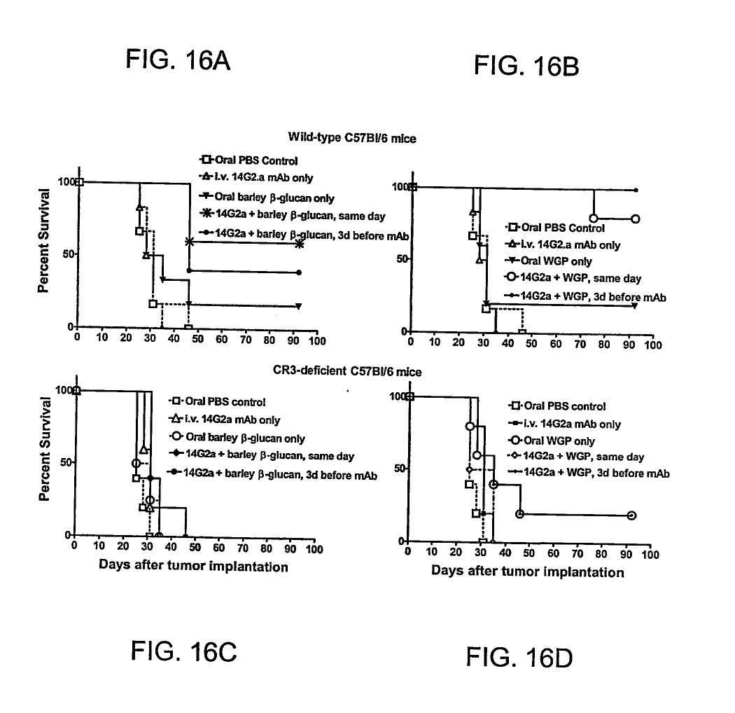

Long-term tumor-free survival with soluble barley or particulate yeast β-glucan therapy requires leukocyte CR3. Results are shown in FIG. 16. This is the survival analysis of the experiment described in FIG. 15.

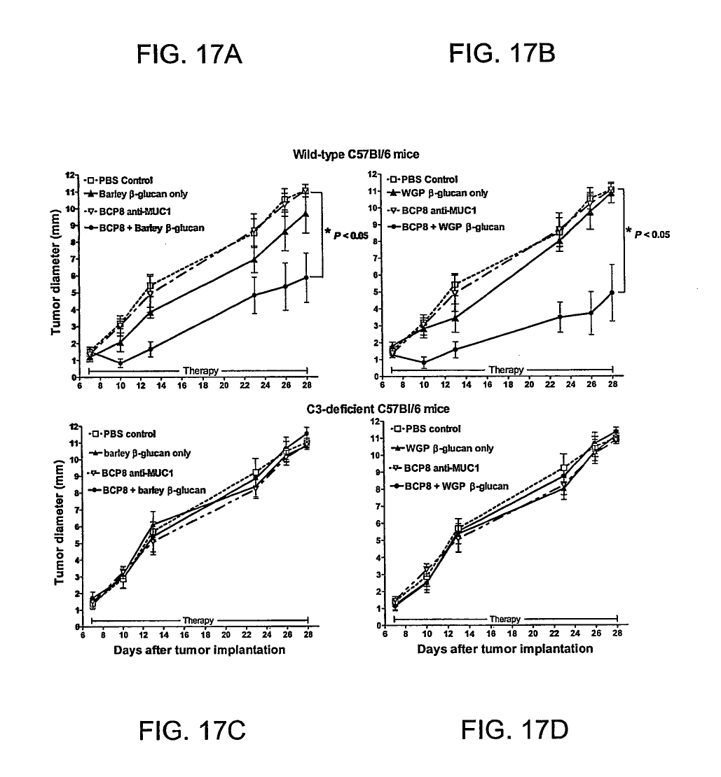

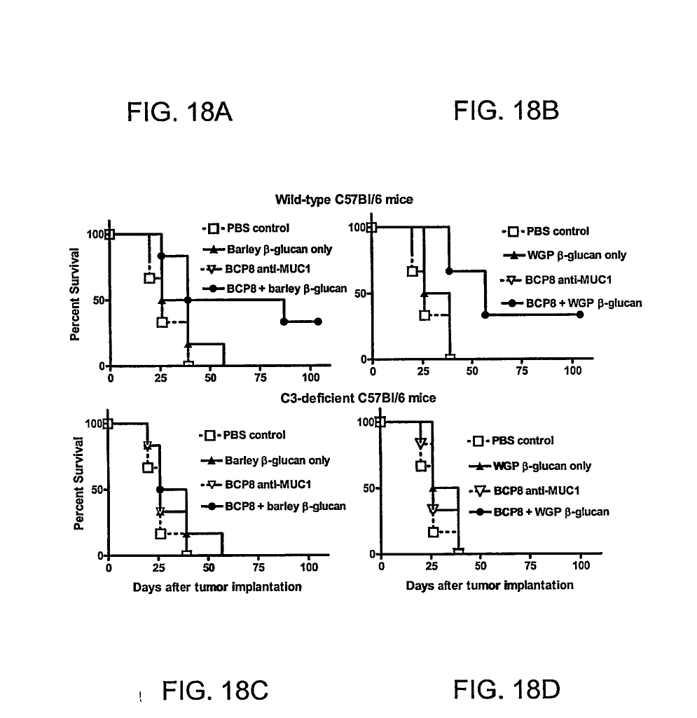

Tumor regression with orally administered soluble barley or particulate yeast β-glucan requires serum C3. As described in Materials and Methods, groups of wild-type or C3-deficient C57B1/6 mice were implanted s.c. with LL/2-MUC1 and after 7 days to allow tumor formation were treated with i.v. BCP8 anti-MUC1 with or without simultaneous oral barley or yeast β-glucan (WGP). Mean values±SD are shown in FIG. 17. Tumor-free survival with orally administered soluble barley or particulate yeast β-glucan requires serum C3. Results are shown in FIG. 18. This is the survival analysis of the experiment described in FIG. 17.

Orally administered fluorescein-labeled particulate yeast β-glucan (WGP-F) is taken up by macrophages that migrate to the spleen, lymph nodes and bone marrow. As described in Material and Methods, wild-type or CR3-deficient C57B1/6 mice were given WGP-F daily by oral gavage and then groups of mice were sacrificed for analysis of the lymphoid organ distribution of cells containing WGP-F by fluorescence microscopy. Results show splenic macrophages isolated from wild-type mice 3 days after daily oral administration of WGP-F. Wild-type and CR3-deficient showed bone marrow macrophages isolated by absorption and elution from fibronectin-coated dishes after 7 days of continuous daily oral administration of WGP-F. There was no detectable difference between wild-type and CR3-deficient bone marrow macrophages in either the proportions of cells containing WGP-F or the size and number of WGP-F per cell. Bone marrow macrophage double-staining with F4/80-Cy5 confirmed that all cells containing ingested WGP-F were macrophages. From days 7 to 12 after daily oral administration of WGP-F, wild-type but not CR3-deficient bone marrow granulocytes were observed that exhibited membrane surface fluorescein staining.

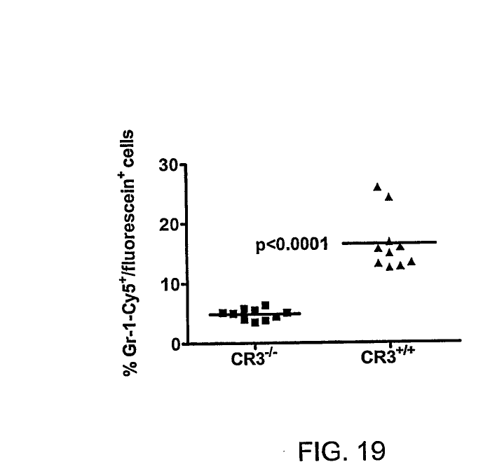

Uptake of soluble β-glucan by the marginated granulocyte pool requires membrane surface CR3. As described in Materials and Methods, 10 wild-type and 10 CR3-deficient C57B1/6 mice were given daily doses of WGP-F by oral gavage for 12 days and then peritoneal granulocytes were elicited with thioglycolate and analyzed for surface fluorescein staining by flow cytometry. For this analysis, granulocytes were identified by double-staining with anti-Gr-1-Cy5. Results are shown in FIG. 19.

In another experiment, wild-type and CR3-deficient mice will be given daily doses of WGP by oral gavage for 7 days. Peritoneal neutrophils will be elicited by overnight treatment of wild-type or CR3-deficient mice with thioglycolate and tested for their ability mediate cytotoxicity of ⁵¹Cr-labeled Ptas64 mammary tumor cells opsonized with iC3b. Neutrophils from mice that had not received oral WGP will served as a control for the effect of the oral WGP. Results will show that thioglycolate-elicited granulocytes from wild-type, but not CR3-deficient mice given oral WGP are able to mediate cytotoxicity of iC3b-opsonized mammary tumor cells in vitro. Granulocytes obtained from wild-type and CR3-deficient mice following 12 days oral administration of WGP will be tested for their ability to mediate cytotoxicity of iC3b-opsonized Ptas64 tumor cells by measuring the release of ⁵¹ Cr.

Those skilled in the art will recognize, or be able to ascertain using no more than routine experimentation, many equivalents to the specific embodiments of the invention described specifically herein. Such equivalents are intended to be encompassed in the scope of the following claims.

We claim:

1. A method of suppressing or eliminating tumor cells, comprising:
administering to a subject in need of treatment a therapeutically effective amount of insoluble β (1,3; 1,6) whole glucan particles and at least one complement activating anti-tumor antibody; wherein the glucan and antibody suppresses or eliminates tumor cells.

2. The method of claim 1, wherein the antibody is introduced via direct administration of a monoclonal or polyclonal antibody or produced via a cancer vaccine.

3. The method of claim 1, wherein the antibody is selected from the group consisting of: trastuzumab, rituximab, cetuximab and combination thereof.

4. The method of claim 1, wherein whole glucan particles and antibody provide a synergistic antitumor effect.

5. The method of claim 1, wherein the whole glucan particles are administered orally.

6. The method of claim 1, wherein the whole glucan particle is administered parenterally.

7. The method of claim 1, wherein the whole glucan particle is derived from yeast.

8. The method of claim 1, wherein the whole glucan particle is derived from a fungal source.

9. The method of claim 8, wherein the fungal source is mushroom.

10. Use of insoluble β (1,3; 1,6) whole glucan particles and complement activating anti-tumor antibody for the manufacture of a medicament for use in treating a neoplastic cell, wherein the combination of glucan and antibody retards the growth of the cell.

11. A method of treating a neoplastic cell comprising administering to said cell a therapeutically effective dose of insoluble β (1,3;1,6) whole glucan particles and a complement activating antibody specific to the neoplastic cell.

12. The method of claim 11, wherein the combination of glucan and antibody retards the rate of growth of the cell.

13. The method of claim 11, wherein the combination of glucan and antibody inhibits the growth of the neoplastic cell.

14. The method of claim 11, wherein the combination of glucan and antibody extends the survival time of a host of the neoplastic cell.

15. The method of claim 1, wherein the complement activating antibody is coated on tumor cells and activates complement via iC3b deposition on the tumor cells.

16. The method of claim 15, wherein the whole glucan particle is taken up by macrophages, degraded and the degraded fragments bind to neutrophils in the bone marrow and through chemotaxis migrate and bind to antibody coated tumor cells where complement has been activated via iC3b deposited the tumor cells.

17. A method of suppressing or eliminating tumor cells, comprising:
administering to a subject in need of treatment a therapeutically effective amount of insoluble β (1,3;1,6) whole glucan particles wherein the whole glucan particles is taken up by macrophages, degraded and the degraded fragments bind to neutrophils in the bone marrow and through chemotaxis migrate and bind to antibody coated tumor cells where complement has been activated via iC3b deposited the tumor cells by a naturally occurring complement activating antibody, wherein the binding of glucan to the iC3b tumor cells results in suppressing or eliminating the tumor cells.

18. A method of suppressing or eliminating tumor cells, comprising:
administering to a subject in need of treatment a therapeutically effective amount of insoluble β (1,3; 1,6) whole glucan particles and at least one complement activating anti-tumor antibody; wherein the glucan and antibody suppresses or eliminates tumor cells and provided that the glucan is not derived from barley.
